(19) United States
(12) Patent Application Publication
(10) Pub. No.: US 2022/0288040 A1
(43) Pub. Date: Sep. 15, 2022

(54) USE OF PYRVINIUM FOR THE TREATMENT OF A RAS PATHWAY MUTATED ACUTE MYELOID LEUKEMIA

(71) Applicants: INSERM (INSTITUT NATIONAL DE LA SANTÉ ET DE LA RECHERCHE MÉDICALE)), Paris (FR); CENTRE NATIONAL DE LA RECHERCHE SCIENTIFIQUE (CNRS), Paris (FR); UNIVERSITÉ TOULOUSE III - PAUL SABATIER, Toulouse (FR); ASSISTANCE PUBLIQUE-HÔPITAUX DE PARIS (APHP), Paris (FR); UNIVERSITÉ DE PARIS, Paris (FR)

(72) Inventors: Jérôme TAMBURINI, Paris (FR); Justine DECROOCQ, Paris (FR); Jean-Emmanuel SARRY, Toulouse (FR); Didier BOUSCARY, Paris (FR); Rudy BIRSEN, Paris (FR)

(21) Appl. No.: 17/639,477

(22) PCT Filed: Aug. 31, 2020
(86) PCT No.: PCT/EP2020/074242

§ 371 Date: Mar. 1, 2022

(30) Foreign Application Priority Data
Sep. 2, 2019 (EP) 19306061.3

(51) Int. Cl. A61K 31/435; A61K 31/519; A61K 45/06; A61P 35/02

(52) U.S. Cl. CPC A61K 31/435; A61K 31/519; A61K 45/06; A61P 35/02

(57) ABSTRACT

Acute myeloid leukemia (AML) are heterogeneous malignancies arising from the multistep transformation of bone marrow immature cells. The inventors showed that RAS pathway mutations were detected in 40% of FLT3- and NPM1-unmutated AML cases and correlated with higher white blood cell count, blast cell percentage and reduced survival after intensive therapy. Building on genetic models of RAS activation, they highlighted the leukemogenic potential of RAS pathway alterations, and the efficacy and limitations of MEK inhibitors in this context. From high-content chemical screens, the inventors unraveled pyrvinium pamoate—an anthelminthic drug approved in human patients—as displaying a preferential cytotoxicity against RAS activated cells. This potential clinical candidate demonstrated a robust synergistic activity with the MEK inhibitor trametinib, including in primary samples from AML patients. Together the data suggest that RAS pathway altered cases may represent a specific AML subtype, in which the anti-leukemic molecule pyrvinium pamoate may represent a new promising therapeutic strategy.

### FIELD OF THE INVENTION

The present invention is in the field of oncology.

### BACKGROUND OF THE INVENTION

Acute myeloid leukemia (AML) are heterogeneous malignancies arising from the multistep transformation of bone marrow immature cells (1,2). Although still associated with a low cure rate, recent advances in our understanding of AML molecular complexity resulted in significant therapeutic improvements for subgroups of patients (3). Particularly, AML harboring mutations in FLT3, IDH1 or IDH2 genes—representing 50% of AML cases—develop an oncogenic addiction to these mutations, offering an avenue for targeted inhibition as recently illustrated by successful tailored clinical trials (4-6). However, many AML cases still lack a druggable oncogenic target, despite the thorough characterization of the molecular landscape of these diseases (7).

Human cancers frequently harbor mutations in the RAS oncogene family including NRAS, KRAS and HRAS, driving oncogenesis through the activation of cellular proliferation and survival (8). RAS are small protein GTPases, regulated by a switch between active GTP-linked and inactive GDP-bound RAS molecules involving a complex network of guanine exchange factors (GEFs, in favor of RAS-GTP) and GTPase activating factors (GAPs, in favor of RAS-GDP). RAS activation—after recruitment by transmembrane tyrosine kinase receptors, or intrinsically in case of activating mutation—elicit the cascade activation of the RAF/MEK/ERK and PI3K/AKT signaling pathways (9). Besides RAS activating mutations conferring independence from physiological regulators, other mutations in genes involved in the RAS network may be found in human cancers such as NF1 (encoding neurofibromin, a RAS GAP), BRAF or PTPN11 (encoding the SHP2 tyrosine phosphatase involved in RAS activation) (9).

Somatic alterations of RAS pathway genes are reported in up to 20% AML cases, notably in NRAS, KRAS, PTPN11 (missense mutations) and NF1 (mutations and deletions) (7,10). Generally arising as secondary driver events, RAS pathway mutations participate to leukemogenesis through mitogen activated protein kinase (MAPK) activation (9,11). The anti-tumor activity of MEK inhibitors in Nras-mutated AML in mice, and in some NRAS or KRAS-mutated AML patients (12,13) suggests that deregulated RAS signaling pathway may represent bona fide targets for therapy. However, currently available strategies mostly involving indirect RAS inhibition are hampered by feedback loops, redundancy and tumor heterogeneity (14-16).

### SUMMARY OF THE INVENTION

As defined by the claims, the present invention relates to use of pyrvinium for the treatment of a RAS pathway mutated acute myeloid leukemia.

### DETAILED DESCRIPTION OF THE INVENTION

The inventors showed that RAS pathway mutations were detected in 40% of FLT3- and NPM1-unmutated AML cases and correlated with higher white blood cell count, blast cell percentage and reduced survival after intensive therapy. Building on genetic models of RAS activation, they highlighted the leukemogenic potential of RAS pathway alterations, and the efficacy and limitations of MEK inhibitors in this context. From high-content chemical screens, the inventors unraveled pyrvinium pamoate—an anthelminthic drug approved in human patients—as displaying a preferential cytotoxicity against RAS activated cells. This potential clinical candidate demonstrated a robust synergistic activity with the MEK inhibitor trametinib, including in primary samples from AML patients. Together the data suggest that RAS pathway altered cases may represent a specific AML subtype, in which the anti-leukemic molecule pyrvinium pamoate may represent a new promising therapeutic strategy.

Thus the first object of the present invention relates to a method of treating a RAS pathway mutated acute myeloid leukemia in patient in need thereof comprising administering to the patient a therapeutically effective amount of pyrvinium.

A further object of the present invention relates to a method of treating a RAS pathway mutated acute myeloid leukemia in a patient in need thereof comprising administering to the subject a therapeutically effective combination comprising MEK inhibitor and pyrvinium.

A further object of the present invention relates to a method of treating a RAS pathway mutated acute myeloid leukemia resistant to MEK inhibitors in a patient in need thereof comprising administering to the subject a therapeutically effective amount of pyrvinium.

A further object of the present invention relates to a method for enhancing the potency of a MEK inhibitor administered to a subject suffering from a RAS pathway mutated acute myeloid leukemia as part of a treatment regimen, the method comprising administering to the subject a pharmaceutically effective amount of pyrvinium in combination with MEK inhibitor.

A further object of the present invention relates to a method of preventing resistance to an administered MEK inhibitor in a subject suffering from a RAS pathway mutated acute myeloid leukemia comprising administering to the subject a therapeutically effective amount of pyrvinium.

As used herein, the term “acute myeloid leukemia” or “acute myelogenous leukemia” (“AML”) refers to a cancer of the myeloid line of blood cells, characterized by the rapid growth of abnormal white blood cells that accumulate in the bone marrow and interfere with the production of normal blood cells.

As used herein, the term “RAS pathway” represents the signalling pathway wherein Ras protein operates. The Ras pathway is well described in the art. Two of the main cellular pathways in which the RAS protein operates are the mitogen-activated protein kinases (MAPK) and phosphoinositide-3 kinase (PI3K) pathways. Typically, the genes involved in the RAS pathway include RAS, NRAS, KRAS, NF1, PTPN11, BRAF, CBL, RASA1, RAF1, SOS1, and MAP2K2.

As used herein, the term “mutation” has its general meaning in the art and refers to a substitution, deletion or insertion. The term “substitution” means that a specific amino acid residue at a specific position is removed and another amino acid residue is inserted into the same position. The term “deletion” means that a specific amino acid residue is removed. The term “insertion” means that one or more amino acid residues are inserted before or after a specific amino acid residue, more specifically, that one or more, preferably one or several, amino acid residues are bound to an a.-carboxyl group or an a,-amino group of the specific amino acid residue.

As used herein, the “RAS pathway mutated acute myeloid leukemia” refers an AML, in which the cancer cells comprise at least one mutation in the RAS pathway. Typically, the patient harbours at least one mutation in at least one gene selected from the group consisting of RAS, NRAS, KRAS, NF1, PTPN11, BRAF, CBL, RASA1, RAF1, SOS1, and MAP2K2. One skilled person can easily identify a mutation in the RAS pathway. For instance, several PCR and/or sequencing based methods are known for use in detecting mutations in the RAS pathway and there exist several commercially available kits (see Dxs Diagnostic Innovations, Applied Biosystems, and Quest diagnostics). In some embodiments, the mutations are identified by next-generation sequencing as described in the EXAMPLE.

As used herein, the term “treatment” or “treat” refer to both prophylactic or preventive treatment as well as curative or disease modifying treatment, including treatment of subject at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “pyrvinium” has its general meaning in the art and refers to the compound having the IUPAC name of:

2-[(E)-2-(2,5-dimethyl-1-phenyl-1H-pyrrol-3-yl)ethenyl]-6-(dimethylamino)-1-methylquinolin-1-ium. Pyrvinium is an anthelmintic effective for pinworms. Several forms of pyrvinium have been prepared with variable counter anions, such as halides, tosylate, triflate and pamoate. In some embodiments, pyrvinium pamoate is used.

A MEK inhibitor is a compound that shows MEK inhibition when tested in the assays titled, “Enzyme Assays” in U.S. Pat. No. 5,525,625, column 6, beginning at line 35. The complete disclosure of U.S. Pat. No. 5,525,625 is hereby incorporated by reference. Specifically, a compound is an MEK inhibitor if a compound shows activity in the assay titled, “Cascade Assay for Inhibitors of the MAP Kinase Pathway,” column 6, line 36 to column 7, line 4 of the U.S. Pat. No. 5,525,625 and/or shows activity in the assay titled, “In Vitro MEK Assay” at column 7, lines 4 to 27 of the above-referenced patent. Alternatively, MEK inhibition can be measured in the assay described in WO 02/06213 A1, the complete disclosure of which is hereby incorporated by reference. MEK inhibitors include, for example, ARRY-142886 (also known as AZD6244; Array BioPharma/Astrazeneca), PD-184352 (also known as CI-1040; Pfizer), XL518 (Exelixis), PD0325901 (Pfizer), PD-98059 (Pfizer), MEK1 (EMD), or 2-(2-amino-3-methoxyphenyl)-4-oxo-4H-[1]benzopyran and 2-(2-chloro-4-iodo-phenylamino)-N-cyclopropylmethoxy-3,4-difluoro-benzamide. Specific preferred examples of MEK inhibitors that can be used according to the present invention include ARRY-142886, PD-184352, PD-98059, PD-0325901, XL518, or MEK1. Specific examples of drugs that inhibit MEK include sorafenib, PD-0325901 (Pfizer), AZD-8330 (AstraZeneca), RG-7167 (Roche/Chugai), RG-7304 (Roche), CIP-137401 (Cheminpharma), WX-554 (Wilex; UCB), SF-2626 (Semafore Pharmaceuticals Inc), RO-5068760 (F Hoffmann-La Roche AG), RO-4920506 (Roche), G-573 (Genentech) and G-894 (Genentech), N-acyl sulfonamide prodrug GSK-2091976A (GlaxoSmithKline), BI-847325 (Boehringer Ingelheim), WYE-130600 (Wyeth/Pfizer), ERK1-624, ERK1-2067, ERK1-23211, AD-GL0001 (ActinoDrug Pharmaceuticals GmbH), selumetinib (AZD6244), trametinib, TAK-733, Honokiol, MEK-162, derivates, and salts thereof.

As used herein the term “resistance to MEK inhibitors” is used in its broadest context to refer to the reduced effectiveness of at least one MEK inhibitor to inhibit the growth of a cell, kill a cell or inhibit one or more cellular functions, and to the ability of a cell to survive exposure to an agent designed to inhibit the growth of the cell, kill the cell or inhibit one or more cellular functions. The resistance displayed by a cell may be acquired, for example by prior exposure to the agent, or may be inherent or innate. The resistance displayed by a cell may be complete in that the agent is rendered completely ineffective against the cell, or may be partial in that the effectiveness of the agent is reduced. Accordingly, the term “resistant” refers to the repeated outbreak of cancer, or a progression of cancer independently of whether the disease was cured before said outbreak or progression.

As used herein, the term “combination” is intended to refer to all forms of administration that provide a first drug together with a further (second, third . . . ) drug. The drugs may be administered simultaneous, separate or sequential and in any order. Drugs administered in combination have biological activity in the subject to which the drugs are delivered. Within the context of the invention, a combination thus comprises at least two different drugs, and wherein one drug is a MEK inhibitor and wherein the other drug is pyrvinium. In some instance, the combination of the present invention results in the synthetic lethality of the cancer cells.

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an antibody of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the agent of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Typically, the drugs of the present invention (i.e. pyrvinium and MEK inhibitor) are administered to the subject in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a subject, the composition will be formulated for administration to the subject. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

The invention will be further illustrated by the following figures and examples. However, these examples and figures should not be interpreted in any way as limiting the scope of the present invention.

### FIGURES

FIG. 1. Identification of pyrvinium pamoate as potential new agent in RAS pathway mutated AML. A. Schematic representation of high-density pharmacological screen in NF1-depleted TF-1 cells. B. First screen with the 1280 compounds at 10 μM using the CellTiter-Glo® cell viability reagent after 72 h of incubation. Results are represented for each compound (identified by a single dot) by the relation between their robust Z-score value (RZ-score) in Y-axis and the percentage of cell growth in X-axis. Compounds with a RZ-score ≤−5 (thus retained for further analysis). C. Second screen performed with serial dilutions of the top-60 compounds from the first screen in NF1-depleted TF-1 cells. Results are presented for each compound illustrated by a dot as the correspondence between their median effective dose (ED50, represented with a Log 10 scale) and drug sensitivity score (DSS). The best hits are highlighted in dark grey, and the classical AML chemotherapies (daunorubicin and cytarabine) are highlighted in light grey. D. Dose-range experiments using log-dilutions (10⁻⁵ to 10⁻⁸ M) of pyrvinium pamoate in CTR, NF1-1.3, NF1-42.8 and NRASG12D Ba/F3 cells. Cell viability was determined using the uptiblue reagent. E-F. Oxygen consumption rate (OCR) and extracellular acidification rate (ECAR) measurement using a seahorse machine in NF1-42.8 or NRAS^{G12D} Ba/F3 cultured with vehicle (CTR) or 250 nM or 500 nM pyrvinium pamoate for 4 h before seahorse analysis. O: oligomycin; F: FCCP (Trifluoromethoxy carbonylcyanide phenylhydrazone); R: Rot/AA (rotenone and antimycin A). E: crude data for OCR and ECAR dependent on time. F: Extrapolation of basal (before oligomycin) and maximal (after FCCP) respiration, and ATP-linked respiration (after oligomycin).

FIG. 2. Synergy between the MEK inhibitor trametinib and pyrvinium pamoate in RAS activated cells. A-B. Synergy scores calculated by the SynergyFinder software (33) using both Bliss and Loewe statistics. A. Results in CTR or RAS activated Ba/F3 or TF-1 cells. B. Results in six primary AML samples harboring RAS pathway mutations. C-D. Colony forming unit leukemia (CFU-L) assays in primary AML samples with RAS pathway mutations incubated with vehicle, 50 nM trametinib, 250 nM pyrvinium pamoate and trametinib/pyrvinium combo during 7 days. Results are presented as a ratio between the number of colonies observed in vehicle-treated cells and each other condition for each sample (indicated by a dot). C. P-value for vehicle/combo comparison is provided on the top of the combo histogram (and not provided for trametinib and pyrvinium comparison with vehicle as not significant). D. Two-by-two comparisons between pyrvinium/trametinib, trametinib/combo and pyrvinium/combo represented with a connecting line between each condition for each single patient sample. Statistical analysis was performed using a Wilcoxon matched-pairs signed rank test.

### EXAMPLE

Material & Methods

Patients

AML patients provided a written informed consent in accordance with the declaration of Helsinki. Blood or bone marrow samples were submitted to a Ficoll-Hypaque density gradient (1800 rpm during 0.5 h) as previously described (17). Mononuclear cells were collected by pipetting, washed once in phosphate buffer saline (PBS), then incubated with a red cell lysis buffer (155 mM NH4Cl, 10 mM KHCO3, 0.1 mM EDTA) for 5 minutes, washed once again in PBS. DNA was immediately extracted using the DNA/RNA Kit (Qiagen, Hilden, Germany) according to manufacturer's procedures. Leftover cells were cryopreserved, and RNA and proteins were extracted shortly after thawing of cryopreserved cells using AllPrep DNA/RNA/Protein Mini Kit (80004, Qiagen, Courtaboeuf, France) according to manufacturer's instructions. Samples containing less than 70% blast cells before Ficoll either were purified using MiniMACS immunoaffinity columns (Miltenyi Biotec, Paris, France) in case of CD34 membrane expression, or sorted with an Aria3 cytometer gating the low side scatter and low CD45-expressing population.

NGS

Targeted sequencing using AmpliSeq™ and Ion Torrent™ technologies: Mutations in selected panels of 30 (RASopathy panel) or 46 (Myeloid panel) genes, or in NF1, EED, EZH2 and SUZ12 genes, were screened by a Next-Generation Sequencing (NGS) assay using the Ion AmpliSeg™ library kit2 384 (Life Technologies, Chicago, Ill.). Multiplex PCR amplifications (233 primer pairs) with panels designed using AmpliSeq™ Designer (version 4.47) on Human genome hg19 were performed from 20 ng of genomic DNA. After amplification, barcodes and adaptors were added to amplicons by ligation. Products were subjected to a selective purification on AMPure beads (Life Technologies). Emulsion PCR (emPCR) was performed using the OneTouchV2 (Life Technologies, Thermo Fisher Scientific, Waltham, Mass., US) instrument. Sequencing was performed on Ion PGM™ (Life Technologies) onto a dedicated 318 V2 chip.

For the “RASopathy” panel, the targeted regions were covered by 390 amplicons of 125-275 bp average length and included the 30 following genes:

| Gene | transcript | location |
|---|---|---|
| SNORD50A | NR 003038 | 6q14.3 |
| SNORD50B | NR 003038 | 6q14.3 |
| SPRY3 | NM 005840 | Xq28 |
| BRAFP1 | NG 003108 | Xq13.3 |
| SPRY1 | NM 001258038 | 4q28.1 |
| PEBP1 | NM 002567 | 12q24.23 |
| MAP2K2 | NM 030662 | 19p13.3 |
| DUSP6 | NM 001946 | 12q21.33 |
| SPRED2 | NM 181784 | 2p14 |
| SPRED3 | NM 001042522 | 19q13.2 |
| SPRY2 | NM 005842 | 13q31.1 |
| SOS1 | NM 005633 | 2p22.1 |
| PTENP1 | NR 023917 | 9p13.3 |
| MAP2K1 | NM 002755 | 15q22.31 |
| HRAS | NM 176795 | 11p15.5 |
| SNHG5 | NR 003038 | 6q14.3 |
| PTEN | NM 000314 | 10q23.31 |
| RRAS | NM 006270 | 19q13.33 |
| SPRED1 | NM 152594 | 15q14 |
| RASA2 | NM 006506 | 3q23 |
| SHOC2 | NM 007373 | 10q25.2 |
| RAF1 | NM 002880 | 3p25.2 |
| BRAF | NM 004333 | 7q34 |
| KRAS | NM 033360 | 12p12.1 |
| ETV5 | NM 004454 | 3q27.2 |
| RASA1 | NM 002890 | 5q14.3 |
| SPRY4 | NM 001127496 | 5q31.3 |
| PTPN11 | NM 002834 | 12q24.13 |
| CBL | NM 005188 | 11q23.3 |
| NRAS | NM 002524 | 1p13.2 |

For the myeloid genes panel, the targeted regions were covered by 606 amplicons of 125-275 bp average length and included the 46 following genes:

| Gene | transcript | location |
|---|---|---|
| ASXL1 | NM 015338 | 20q11.21 |
| BCOR | NM 001123385 | Xp11.4 |
| BCORL1 | NM 021946 | Xq26.1 |
| BRAF | NM 004333 | 7q34 |
| CALR | NM 004343 | 19p13 |
| CBL | NM 005188 | 11q23.3 |
| CSF3R | NM 156039 | 1p34.3 |
| CSNK1A1 | NM 001025105 | 5q32 |
| CUX1 | NM 001202543 | 7q22.1 |
| DDX41 | NM 016222 | 5q35.3 |
| ETNK1 | NM 018638 | 12p12.1 |
| DNMT3A | NM 022552 | 2p23.3 |
| ETV6 | NM 001987 | 12p13.2 |
| EZH2 | NM 001987 | 7q36.1 |
| FLT3 | NM 004119 | 13q12.2 |
| GATA2 | NM 001145661 | 3q21.3 |
| HRAS | NM 001130442 | 11p15.5 |
| IDH1 | NM 005896 | 2q34 |
| IDH2 | NM 002168 | 15q26.1 |
| JAK2 | NM 004972 | 9p24.1 |
| KDM6A | NM 021140 | Xp11.3 |
| KIT | NM 000222 | 4q12 |
| KRAS | NM 033360 | 12p12.1 |
| MPL | NM 005373 | 1p34.2 |
| MYD88 | NM 001172567 | 3p22.2 |
| NRAS | NM 002524 | 1p13.2 |
| PHF6 | NM 001015877 | Xq26.2 |
| PPM1D | NM 003620 | 17q23.2 |
| PTEN | NM 000314 | 10q23.31 |
| PTPN11 | NM 002834 | 12q24.13 |
| RAD21 | NM 006265 | 8q24.11 |
| RHOA | NM 001664 | 3p21.31 |
| RIT1 | NM 006912 | 1q22 |
| RUNX1 | NM 001754 | 21q22.12 |
| SETBP1 | NM 015559 | 18q12.3 |
| SF3B1 | NM 012433 | 2q33.1 |
| SH2B3 | NM 005475 | 12q24.12 |
| SRSF2 | NM 001195427 | 17q25.1 |
| STAG2 | NM 001042749 | Xq25 |
| STAT3 | NM 139276 | 17q21.2 |
| TET2 | NM 001127208 | 4q24 |
| TP53 | NM 000546 | 17p13.1 |
| U2AF1 | NM 001025203 | 21q22.3 |
| WT1 | NM 024426 | 11p13 |
| ZRSR2 | NM 005089 | Xp22.2 |

NF1, EZH2, EED and SUZ12 were sequenced as previously described (18,19)

Bio-informatics analysis of sequencing data: Base calls were generated by the Torrent Suite™ Software (v. 5.6) using the included variant caller with an additional plug-in (Life Technologies). The .bam and .vcf files were used for analysis. Detection of single nucleotide variations (SNVs) and short insertions/deletions from the BAM files was performed using the Torrent Suite Variant Caller (TSVC) plugin from the Torrent Suite Software v5.0.4 (Thermo Fisher Scientific, Waltham, Mass., US). The .vcf files were annotated with the Ion reporter software (Life Technologies) and processed for a second analysis of the indexed files using the NextGENe software (Softgenetics, State College, Pa.). Results were compared to select abnormalities that will be further considered. Filtered candidate variants listed in TSVC files were then annotated, ranked, and interpreted using the Polydiag suite (Bioinformatics Department, Paris-Descartes University). Moreover, aligned reads from BAM files were visualized using the Integrative Genomics Viewer v2.3 from the Broad Institute (Cambridge, Mass., USA). Assessment of variants implication was performed based on population databases (dbSNP and GnomAD), mutation databases (COSMIC), and predictions software (Alamut, mutation taster, OncoKB, and Cancer Genome Interpreter).

Fluorescence In Situ Hybridization (FISH)

Dual color FISH experiments were performed using a XL TP53/NF1 D-5089-100-OG probe (Metasystems probes, Altlussheim, Germany), targeting a 167 kb region of TP53 (probe labeled with Rhodamine-dUTP) and a 312 kb region of NF1 (probe labeled with FITC-dUTP). Hybridization was performed as described previously (20). The images were captured by a CCD camera fixed on a BX61 microscope (Olympus, Rungis, France), and processed with a Case data Manager 6.0 software (Applied Spectral Imaging).

RT-qPCR

RNA quality was evaluated with a Bioanalyzer 2100 (using Agilent RNA6000 nano chip kit). RNA was and retrotranscribed into cDNA. qPCR was performed using SYBR Green I master mix on LC480 (Roche, Bale, Swiss). For quantification, CP values were used to calculate the normalized ratio quantities (NRQ) according to the formula: NRQ=RQ/NF (RQ: E^{ΔCP}; ΔCP: difference between the CP of the gene of interest and the mean CP of this gene in all samples; E: primer efficacy; NF: mean RQ of the reference genes). Results were expressed as NRQ to reference genes B2M, UBC and ACTB.

Cell Lines and Reagents

We used the TF-1 AML cell line, which was identified by PCR-single-locus-technology (Promega, PowerPlex21 PCR Kit, Eurofins Genomics). Cells were cultured in RPMI 1640 medium (Gibco 61870; Life Technologies, Saint Aubin, France) supplemented with 10% FCS, 2 mM glutamine (Gibco 25030; Life Technologies, Saint Aubin, France), 100 IU/mL penicillin and 100 μg/mL streptomycin (Gibco 15140; Life Technologies, Saint Aubin, France) at 37° C. under a 5% CO₂ atmosphere. TF-1 cells were cultured with 5 ng/mL of human GM-CSF (130-093-866, Miltenyi Biotec, Paris, France). We also used the BaF/3 murine hematopoietic cell line cultured with IL-3 provided by a conditioned medium harvested from cultured WEHI-3 cells (21). Trametinib (GSK1120212) was purchased from Selleck chemicals LLC (Houston, USA) and Pyrvinium pamoate (P0027) was from Sigma Aldrich-Chimie (Saint Quentin Fallavier, France). Chemical compounds for the repurposing screen were purchased from Prestwick Chemicals V3 (a unique collection of 1,280 small molecules, mostly approved drugs FDA, EMA and other agencies) and obtained in Dimethyl Sulfoxide (DMSO) as 10 mM stock solution.

Constructs

CRISPR/Cas9: Human and murine NF1-targeting guide RNA were designed using the Optimized Crispr Design application from the laboratory of Dr Feng Zhang (http://crispr.mit.edu/, no longer available) as previously described (17). The human guides were then cloned into the plentiCRISPRv1 puromycin plasmid (#49535 no longer available, Addgene) (22) while the murine guides were cloned into the plentiCRISPRV2 mCherry plasmid (LentiCRISPRv2-mCherry was a gift from Agata Smogorzewska (Addgene plasmid #99154; http://n2t.net/addgene:99154; RRID:Addgene_99154).

NRAS G12D: Hs NRAS G12D in pDonor-255 (Hs.NRAS G12D was a gift from Dominic Esposito (Addgene plasmid #83176; http://n2t.net/addgene:83176; RRID:Addgene_83176) was cloned into the plenti PGK puro DEST (pLenti PGK Puro DEST (w529-2) was a gift from Eric Campeau & Paul Kaufman (Addgene plasmid #19068; http://n2t.net/addgene:19068; RRID:Addgene_19068) (23)) using the Gateway system (Life Technologies, Carlsbad, Calif., USA).

Lentivirus Production and Cell Line Infections

Lentivirus production and cell line infections were done as previously described (24). Briefly, we used 293-T packaging cells to produce all of the constructed recombinant lentivirus through co-transfection of these cells with the packaging plasmids pMD2.G and psPAX2 encoding lentiviral proteins (Gag, Pol, and Env) using Lipofectamine 2000 Transfection Reagen (Thermo Fischer Scientific, Waltham, Mass., US). Supernatants were collected and ultracentrifuged for 48 h after transfection over two consecutive days, and then stored at −80° C. AML cell lines were seeded at 2×10⁶/ml and 10 μl of lentiviral supernatants were added for 24 h. Cells were further selected with puromycin, or cell sorted with an ARIA 3 cytometer in case of GFP or mCherry expression as selection marker.

Immunoblots

Cells were lysed in 100 μL 1× Laemmli buffer [62.5 mM Tris HCl pH 6.7, 10% glycerol, 2% sodium dodecylsulfate (SDS), 24 mM dithiotreitol (DTT), 2 mM Vanadate, bromophenol blue], heated at 90° C. for 5 min and resolved by SDS-polyacrylamide gels electrophoresis, transferred to nitrocellulose membranes, and probed with primary antibodies. Protein signals were revealed by chemoluminescence (ECL, Bio-Rad, Marnes la coquette, France) and detected using a CCD camera (LAS 3000 Fujifilm, Tokyo, Japan). Primary antibodies used were directed against: β-actin (#A1978, Sigma Aldrich, Saint-Louis, Mo., US), HSC70 (#7298, Santa Cruz Biotechnology, Dallas, Tex., US); phospho-AKT T308 (#4056, Cell Signaling Technology (CST), Danvers, Mass., US), phospho-ERK 1/2 T202-Y204 (#4377, CST, Danvers, Mass., US), phospho-STATS Y694 (#9351, CST, Danvers, Mass., US), NF1 (#14623, CST, Danvers, Mass., US), RAS (#05-016, Merck Millipore, Burlington, Mass., US), PI3K p85α (#423, Santa Cruz Biotechnology, Dallas, Tex., US), dived Caspase 3 (#9661, CST, Danvers, Mass., US), PARP (#9542, CST, Danvers, Mass., US).

RAS Pull Down Assay

RAS activity was assessed by a GST-RAF1-RBD pull down assay according to manufacturer's instruction (17-218, Merck Millipore, Burlington, Mass., US). Briefly, 5×10⁷ cells were lysed and active RAS was pulled down after interaction with a RAF1-RBD motif conjugated with agarose beads. Beads were then solubilized in Laemmli buffer and RAS detection—proportional to its activity unraveled by the RAS-RAF interaction—was performed by immunoblotting.

Trypan Blue Dye Exclusion Assay

The Trypan Blue dye (Sigma Aldrich, Saint Quentin Fallavier, France) exclusion assay was used to determine the number of viable cells present in the cell suspension. A Malassez counting chamber was filled with the cell suspension mixed with the dye. Cells were then visually examined and counted under a microscope: cells taking up the dye were considered dead and cells excluding the dye were considered alive.

Contact-Free Cell Co-Culture

TF1 NF1-1 and TF-1 NF1-2 cells were seeded at 3×10⁵/mL without GM-CSF. The next day, Corning transwells (Merck, Merck Millipore, Burlington, Mass., US) were inserted on the top of milieu alone well (control), NF1-1 and NF1-2 TF-1 cells wells, and filled with TF-1 CTR cells in the absence of GM-CSF. Trypan blue exclusion assays were carried out on days 1, 2, 3 and 6.

Gene Expression Profiling

RNA quality was evaluated with a Bioanalyzer 2100 (using Agilent RNA6000 nano chip kit), and 100 ng of total RNA was reverse transcribed using the GeneChip® WT Plus Reagent Kit according to the manufacturer's instructions (Affymetrix, Thermo Fischer Scientific, Waltham, Mass., US). Briefly, double strand cDNA was used for in vitro transcription with T7 RNA polymerase and 5.5 μg of Sens Target DNA were fragmented and labelled with biotin. The cDNA were then hybridized to GeneChip® Clariom S Human (Affymetrix, Thermo Fischer Scientific, Waltham, Mass., US) at 45° C. for 17 hours, then washed on the fluidic station FS450 (Affymetrix, Thermo Fischer Scientific, Waltham, Mass., US), and scanned using the GCS3000 7G (Thermo Fischer Scientific, Waltham, Mass., US). Scanned images were then analyzed with Expression Console software (Affymetrix, Thermo Fischer Scientific, Waltham, Mass., US) to obtain raw data (.cel files) and metrics for quality controls. Raw fluorescence intensity values were normalized using Robust Multi-array Average (RMA) algorithm in R to generate the normalized data matrix by performing background correction, quantile normalization and log 2 transformation of raw fluorescence intensity values of each gene. All quality controls and statistics were performed using Partek® Genomics Suite software (Partek, St. Louis, Mo., USA). Data were normalized using custom brainarray CDF files (v20 ENTREZG). To identify differentially expressed genes, we applied a classical analysis of variance (ANOVA) with a FDR permutation-base for each gene. We created a new matrix with only the significant ANOVA site and performed Z-scoring of rows. Hierarchical clustering by Pearson's dissimilarity and average linkage and principal components analysis (PCA) were conducted in an unsupervised fashion to control for experimental bias or outlier samples. We set a filter for those genes that displayed at least a ≥1.5 or ≤−1.5 fold difference in expression between groups and achieved an FDR of <0.05. Data were then interrogated for evidence of biologic pathway dysregulation using Gene set enrichment analysis (GSEA, Broad Institute). Enrichment rates were considered significant when the P-value <0.05 and the FDR ≤0.1.

TF-1 Differentiation

Cells were washed 3 times in PBS to remove GM-CSF, and then cultured 7 days with 2 IU/mL EPO. Cells were spin down to collect pellets in which color change from white to purple reflected hemoglobinization.

Cell Line Derived Xenografts Experiments

Cell line derived Xenografts experiments were done as previously described (25). All animal studies were conducted in accordance with the guidelines of the Association for Assessment and Accreditation of Laboratory Animal Care International and with approval of the local ethics committee, as reported (21). Adult NSG mice (6-8 weeks old) were treated with 20 mg/kg busulfan (Busilvex, Pierre Fabre, France) by intraperitoneal administration 24 h before injection of leukemic cells. TF-1 AML cell lines were washed twice in PBS and cleared of aggregates and debris and suspended in PBS at a final concentration of 2×10⁶ cells in 200 μl of PBS per mouse. AML cells were xenografted in the tail vein of mice. In some experiments, mice were treated with 0.5 mg/kg/d Trametinib per oral gavage in corn oil containing 4% final volume of DMSO 5/7 days since day 8 post graft, or with vehicle. Daily monitoring of mice determined the time of killing (usually ruffled coat, hunched back, weakness and reduced motility).

Immunohistochemistry

Femurs, tibias and spleens of mice were fixed for 24 h in 4% paraformaldehyde. Decalcification was carried out using 15% formic acid at 4° C. for 4 h, followed by a second fixation in 4% paraformaldehyde during 24 h. Samples were paraffin embedded and then sliced using a microtome. Four μm thick serial sections were analyzed by immunohistochemistry using anti-phospho-ERK antibody (#4370, CST, Danvers, Mass., US) with Immunohistochemistry Application Solutions Kit (Rabbit) (#13079, CST, Danvers, Mass., US) according to the manufacturer's instructions. For antigen retrieval, slides were heated in citrate buffer (10 mM sodium citrate buffer pH 6.0) for 10 min. Primary antibodies were used at the dilution 1/200, in antibody diluent (#8112, CST, Danvers, Mass., US) for anti-phospho-ERK antibody (#4370, CST, Danvers, Mass., US) and incubated over night at 4° C. Detection of primary antibodies was carried out using the Signal Stain Boost IHC Detection Reagent (#8114, CST, Danvers, Mass., US) and Signal Stain DAB Substrate (#8059, CST, Danvers, Mass., US) based on conversion of diaminobenzidine to a dye with multimeric horseradish peroxidase (HRP). Sections were counterstain with Hematoxylin. Images were acquired and processed using the slide scanner and software Zeiss Axioscan.Z1 (Carl Zeiss AG, Oberkochen, Germany).

Cell Viability Assays

UptiBlue: Cells were seeded in 100 μl of culture medium for 48 hours. Cell density was different between cell lines (2×10⁵/ml) and primary samples (10⁷/ml) due to differences in metabolic activities and proliferation rates, which significantly influenced signal detection. The UptiBlue viable cell-counting reagent (Interchim, Montluçon, France) was then added for 4 hours and fluorescence was measured with a Typhoon 8600 scanner (GE Healthcare BioSciences, Buc, France).

CellTiter-Glo 2.0 Assay: A robot distributed 25 μl of the CellTiter-Glo 2.0 Assay reagent (Promega Inc., Madison, USA) in each well containing cells of a 384-well plate. The contents were mixed for 2 minutes at 300 rpm on an orbital shaker (Titramax 100, Dutscher, Issy-les-moulineaux, France) and plates were incubated for 10 minutes at room temperature to stabilize luminescent signals. Units of luminescent signal generated by a thermo-stable luciferase are proportional to the amount of ATP presented in viable cells. Luminescence was recorded using a CLARIOStar (BMG Labtech, Ortenberg, Germany) reader at a gain of 3600.

Flow Cytometry

Apoptosis was measured using Alexa fluor 647-coupled annexin V (#A23204, Thermo Fisher Scientific, Waltham, Mass., US). Data were generated on an LSRFortessa apparatus (BD Biosciences, le pont de claix, France) and analyzed using Kaluza software (Beckman Coulter, Miami, Fla.).

Target Selective Inhibitor Library Screen

We used the target selective inhibitor library solubilized in DMSO at a final concentration of 10 μM distributed in 96 wells plates to screen TF-1 CTR, NF1-1 and NF1-2 cells using the uptiblue cell viability reagent as described above. This screen was performed three times separately. After background noise subtraction, outliers were removed, and data were normalized and presented as a percentage of the conditions incubated with the vehicle (DMSO).

Prestwick Chemical Library® (PCL) Screen

Cells were seeded in 384-well plates (ViewPlate-384 Black—Perkin Elmer, ref 6007460) using a MultiDrop combi (Thermo Fisher Scientific, Waltham, Mass., US), in 40 μL of cell media at 37° C. for 24 h. Cells densities per well were determined as follows: 5×10³ for TF-1 CTR and TF-1 NF1-1 and 6×10³ for TF-1 NF1-2 using T4 Cellometer (Nexcelom).

Primary Screening

We used the 1280 compounds of the PCL at 10 μM (in 0.5% DMSO) delivered to the using the MultiChannel Arm™ 384 (MCA 384) (TECAN, Männedorf, Swiss). The plates were incubated 72 h at 37° C. in 5% CO2 before assaying cell viability using the CellTiter Glo® reagent and luminescence detection as described above.

Secondary Screening

We selected the top 60 hits from our primary screen to perform a set of dose-range experiments using the same workflow. We performed three independent experiments using 8 consecutive three-fold dilutions from 10⁻⁶M to 4.57×10⁻⁹M. Both primary and secondary screens were performed on the same batches of viably frozen cells and at the same passage stage (four passages from thawing). We used the CellTiter-Glo 2.0 Assay kit (Promega Inc., Madison, USA) to assess cell viability.

Data Processing

Values of all plates were visually inspected for systematic bias (i.e., edge effects). Measurements data were analyzed using software developed by the Biophenics platform (Curie Institute, Paris, France). For hit identification, we use the robust Z-score method under the assumption that most compounds are inactive and can serve as controls (26,27). In order to correct for plate positional effects, an automated iterative median filtering was developed. Luminescence intensity raw values were first log 2 transformed to make the data more symmetric and close to a normal distribution. Next, median polishing (27) was applied to progressively corrects columns, rows, and entire plates by subtracting their median, repeating until convergence of values. In our implementation, column and row corrections were computed separately for replicates, but across all plates within the replicate in combination. Hits for each compound were identified as follows: sample median and median absolute deviation (MAD) were calculated from the population of screening data points (named as sample) and used to compute robust Z-scores (Iglewicz and Hoaglin, 1993) according to the formula:

“RZ-score”=(x−sample median)/(1.4826×MAD)

where x corresponds to the drug-treated data point and MAD is the median of the absolute deviation from the median of the tested wells. A compound was identified as a hit if the RZ-score was <−2 or >2 pointing in the same direction in both replicates. Compounds having a RZ-score <−2 corresponds to those considered reducing cell viability. The same analysis pipeline was applied to each cell lines tested. Final values correspond to the mean RZ-score for each compound.

In dose-range experiments, compound activity was normalized on a per-plate basis by dividing the value in each well by the median value of the control wells (100% cell viability). For each compound, a four parameters log-logistic model was then fitted on the pooled replicate data with the R package drc (28). Compound activity was then summarized by computing a Drug Sensitivity Score (DSS, modified from (29)), the area under the curve normalized by the area of an inactive compound (100% viability at all doses). Finally, we scored these compounds by calculating their ED50×DSS value and we focused on the top-10 compounds among which were cytarabine and daunorubicin.

Mitostress Analysis

Oxygen consumption rate (OCR) and extracellular acidification rate (ECAR) were measured using a Seahorse XF96 extracellular flux analyzer (Seahorse Bioscience, North Billerica, Mass., USA), as reported (30). Briefly, 1.5×10⁵ cells were seeded in 96-well XF96 well plates coated with BD Cell-Tak (Becton Dickinson Biosciences, Franklin Lakes, N.J., USA) and loaded with XF Dulbecco's Modified Eagle's Medium. After 1 h incubation at 37° C. without CO2, cells were transferred to the XF96 analyzer, and OCR and ECAR were measured. Oligomycin (1 μM) was added after 20 min, followed by FCCP (2 μM) after 40 min and Antimycin A/Rotenone (1 μM) after 59 min.

Wnt Reporter Activity Assay

HuH6 cells were seeded at 3×10⁵ in 1004, and TF-1 cells (CTR, NF1-1, NF1-2 and NRAS^{G12D}) at 10⁶ in 100 μL and incubated without or with 250 or 2500 nM pyrvinium pamoate for 16 h. Then, cells were transfected with the TCL/LEF-Firefly luciferase and Renilla luciferase expression vectors, as reported (31) using Lipofectamine 3000 reagent (Thermo Fisher Scientific, Waltham, Mass., US) according to manufacturer's instructions. Cells were collected 16 h after transfection and luciferase activity was measured using the Dual-Luciferase Reporter Assay System (Promega, Charbonnières-les-bains, France) and a Clariostar plus microplate reader (BMG Labtech, Ortenberg).

Leukemia Colony Forming Units (CFU-L) Assay

CFU-L assays were performed as previously described (32). Briefly, AML cells were seeded at 10⁶/ml in H4230 medium (StemCell Technologies, Vancouver, Canada) supplemented with 10% of conditioned medium harvested from cultured 5637 cells. At day 7, CFU-L (colony of >20 cells) were scored under an inverted microscope.

Synergistic Cell Viability Assays

We performed dose-range experiments of trametinib and pyrvinium single-agents or combination and assessed cell viability after 48 h using the uptiblue reagent. We used the SynergyFinder online software to calculate synergy scores computed using the zero interaction potency (ZIP) model (33). All experiments were done three times separately and pooled data were analyzed.

Statistics

Differences between the mean values obtained for the experimental groups were analyzed using the two-tailed Student's t test or a Mann-Withney test in case of non-parametric data. Two by two comparisons between colonies ratio of matched-pairs samples were made by using a Wilcoxon matched-pairs signed rank test. Statistical analysis of categorical variables was performed using the Chi-2 test or the Fisher exact test in case of non-parametric data. Survival curves analysis was performed using Log Rank (Mantel Cox) test. Statistical analyses were performed using Prism software 8.1.1 (GraphPad). Vertical bars indicate standard deviations. *P<0.05, **P<0.01, ***P<0.001.

Results

RAS Pathway Gene Mutations Landscape in AML.

We performed next-generation sequencing (NGS) in genomic DNA samples from 127 AML patients for a panel of genes whose variants are associated with genetic inherited syndromes characterized by RAS activation, referred to as RASopathies, and also mutated in cancer (data not shown) (9,34). To focus on AML cases with unmet need for new efficient therapies, we excluded patients with favorable cytogenetic features, harboring t(8;21), inv(16) or t(15;17) abnormalities, as well as those with CEBPA biallelic mutations (data not shown). Moreover, on an analysis of the Cancer Genome Atlas (TCGA) data, FLT3 mutations appeared exclusive from RAS pathway mutations, and FLT3-mutated cases were excluded from our cohort (data not shown). While associated with RAS mutations in 30% of AML cases (data not shown), we excluded cases with NPM1 mutations as associated with a seemingly favorable prognosis when co-occurring with RAS mutations (7,35). Based on TCGA data, we retained for RASopathy NGS analysis approximately 46% of all AML cases (data not shown).

Our initial cohort was constituted of 140 AML patients, including 127 cases and 13 controls (inv(16), N=3; t(8;21), N=3; NPM1/FLT3-ITD, N=6; NPM1, N=1) (data not shown). We applied our NGS RASopathy panel to these 140 samples and sequencing data were available in 135 (data not shown, technical failure occurred in five cases, NRAS and KRAS genes were sequenced using the Sanger method in two cases). NF1 and polycomb repressor 2 (PRC2) members (SUZ12, EZH2, EED) genes were sequenced using a dedicated NGS panel, and copy number variations (CNVs) were assessed to detect deletions (Missing data in 12 cases, data not shown). Moreover, deletions at the NF1 locus were controlled by fluorescent in situ hybridization (FISH) in 104 samples (data not shown).

We detected at least one RAS pathway gene alteration in 50 (40%) AML samples from our 127 cases (data not shown). NF1 mutations/deletions were found in 17 cases (14.8%), while NRAS, KRAS, PTPN11, CBL and BRAF were detected in 13 (10.4%), 10 (8%), 9 (7.2%), 5 (4%) and 2 (1.6%) cases, respectively (data not shown). RAF1, RASA1, SOS1 and MAP2K2 mutations were detected in a single case each in our cohort data not shown). Patients with RAS pathway mutations harbored slightly more adverse cytogenetic feature and adverse ELN scores (data not shown).

NF1 alterations were 3 missense mutations, 3 frameshift mutations, one splice-site mutation, and 11 deletions including three only detected by FISH (data not shown). One patient had a NF1 mutation associated with a NF1 deletion. These alterations were more frequently detected in complex karyotype samples (57% of NF1-mutated and 90% of NF1-deleted cases, data not shown). Genes encoding members of the histone methyl transferase polycomb repressor complex 2 (PRC2) are frequently subject to loss of function mutations in NF1-altered tumors such as juvenile myelomonocytic leukemia (JMML) and malignant peripheral nerve sheath tumors (MPNSTs)(18,36). Similarly, we observed in NF1-altered, an increased prevalence of deletions/mutations of SUZ12, EZH2 and EED genes (data not shown).

We observed a co-occurrence of RAS pathway mutations in nine samples, mostly concerning NRAS, KRAS and PTPN11 (data not shown). For two of these samples, hypothesis concerning their clonal architecture may be proposed (data not shown). In sample #50, two different variants of PTPN11 (G503V and D61Y) were detected at a similar variant allele frequency (VAF, 21% and 18%, respectively) along with del(9q) karyotype, and DNMT3A and KDM6A mutations at a 91% and 100% VAFs, respectively. These data suggested the occurrence of either two different PTPN11 subclones, or a single clone with two PTPN11 variants inside the main KDM6A/DNMT3A clone (data not shown). Sample #155 is of particular interest, as six different RAS-mutated subclones (five different NRAS and one KRAS mutations) were detected at low VAFs inside a large STAG2/GATA2/RUNX1 clone, supporting the notion of clonal interference in this sample, as reported in a significant fraction of RAS-mutated t(8;21) and inv(16) AML (data not shown). In the remaining cases for which a clonal architecture may be proposed, RAS pathway mutations may have occurred within the dominant clone (samples #56, #201 and #183), or lately as subclones (sample #24). These data suggested that RAS pathway mutations may be present in the main clone, or may occur lately as subclonal events in the course of AML oncogenesis.

Together our data describe the repartition of RAS pathway mutations in a large focused cohort of AML patients without actionable therapeutic target.

Prognostic Impact of RAS Pathway Gene Mutations in AML.

The prognostic value of RAS pathway alterations considered as a whole has not been evaluated in AML. From our main cohort, we retained for analysis a homogeneous group of 91 patients intensively treated with cytarabine plus anthracyclin-based induction chemotherapy (data not shown).

While gender and age, as well as the proportion of secondary AML were similar between RAS pathway mutated and other cases, patients with RAS mutations had a significantly higher white blood cell count (WBC), percentage of blood- and bone marrow-infiltrating blast cells and lactate dehydrogenase (LDH) levels (Table 1). Notably, both groups had the same proportion of refractory disease and completion of allogenic hematopoietic stem cell transplantation (Table 1). These data suggested that RAS pathway altered AML cells had higher proliferation capacities in patients.

Among this cohort, survival proportions were concordant with other series (37), and widely used prognostic markers including Medical Research Council (MRC) cytogenetic categories and ELN score discriminate the patients as reported (data not shown). When considering the whole cohort, the detection of RAS pathway mutations correlated with a reduced overall survival probability, while having no impact on progression-free survival (data not shown). Focusing on ELN intermediate patients, RAS pathway mutations were predictive of reduced progression-free and overall survival probabilities, in contrast to our observations for the ELN adverse group (data not shown). The adverse prognostic of RAS pathway alterations was not observed in our analysis of the TCGA and BEAT AML databases, in which however fewer RAS-related abnormalities were detected (data not shown). While not correlated to survival considering the whole cohort (data not shown), NRASG12/Q61R mutations had a near significant correlation with a better survival probability compared to other RAS pathway alterations, which was not found with KRAS or PTPN11 mutations (p=0.055, data not shown).

We further hypothesized that NF1 gene expression may represent a clinically relevant variable. We evaluated NF1 mRNA abundance by quantitative PCR in 54 AML samples, and observed variable levels of NF1 expression with a mean expression of 1.27 (range: 0.11-4.42, data not shown). Among these cases, 34 were homogeneously treated by intensive chemotherapy and displayed a similar NF1 gene expression pattern (data not shown). While low and high NF1-expressing patients had similar survival proportions overall, low NF1 expression significantly discriminated a subgroup of patients with reduced survival among the RAS pathway mutated cases (data not shown).

These data suggested that RAS pathway alterations were associated with increased proliferation potential, and correlated with reduced survival probability in AML, particularly within the ELN intermediate group.

Development and Characterization of AML Cell Line Models of RAS Activation.

We used cytokine-dependent cell lines to establish the oncogenic potential of RAS pathway genetic modifications (38,39). TF-1 and UT-7 are human AML cell line requiring granulocyte-macrophage colony stimulating factor (GM-CSF) or erythropoietin (EPO), respectively, to proliferate and survive in vitro. The Ba/F3 murine cell line established from normal pro-B cells is dependent on interleukine-3 (IL3) (40,41). After cytokine starvation, parental cell lines undergo cell cycle arrest and apoptosis, while cells modified with an oncogenic signal exponentially grow in the absence of cytokines (data not shown).

First, we used NF1-targeting CRISPR/Cas9 to deplete TF-1, Ba/F3 and UT-7 cell lines from neurofibromin. After lentiviral transduction, cell lines were starved from cytokines and while the control-transduced cells declined within a week, CRISPR-modified cells grew readily from a bulk population, in contrast to control cells (data not shown). NF1 knockdown was clearly observed in TF-1 and Ba/F3 cells, but not in UT-7 cells having a low-to-no NF1 baseline protein detection (data not shown). Moreover, an increased ERK phosphorylation attested for RAS activation in NF1 CRISPR cells compared to controls (CTR) in all three cell lines, which was confirmed by RAS-RAF1 pulldown assays (data not shown). We also transduced TF-1 and Ba/F3 cells with a vector allowing the expression of NRAS^{G12D}, which also strongly induced RAS activity and ERK phosphorylation (data not shown).

We performed a gene expression-profiling assay in NF1 knockdown TF-1 cells, compared to CTR TF-1 cells (labelled NF1^{KD} and NF1^{WT}, respectively), after 6 h of GM-CSF starvation. Using gene set enrichment analysis (GSEA) (42,43), number of RAS-related gene sets were scored among the most significant normalized enrichment scores in NF1-depleted cells (data not shown), which confirmed the strong activation of RAS pathways achieved by NF1 knockdown in these cells. Moreover, we took advantage of the EPO-induced differentiation capacity of TF-1 cells (44), and observed a marked hemoglobinization of CTR cells—a hallmark of erythroid differentiation—which was absent in NF1 knockdown cells in long-term culture with EPO, suggesting that RAS activation blocked the differentiation program to favor proliferation in NF1-depleted cells (data not shown). These data showed that NF1 depletion induced a strong RAS activation signature in TF-1 cells.

We observed that cytokine starvation allowed the continuous growth of NF1 knockdown TF-1, Ba/F3 and UT-7 cells, as well as TF-1 and Ba/F3 expressing NRAS^{G12D}, which contrasted with the absence of proliferation in control cell lines upon starvation (data not shown). To exclude an autocrine production of pro-survival cytokines in RAS-activated cells, we performed contact-free cell co-culture experiments, in which TF-1 CTR cells were cultured alone, or with GM-CSF-free TF-1 NF1-1 or TF-1 NF1-2 cells. As TF-1 CTR cells showed no proliferation when exposed to cytokines produced by NF1 knockdown cell (data not shown), we concluded that the cytokine-independent capacities acquired upon NF1 depletion were not related to an autocrine/paracrine cytokine production but rather due to a cell-autonomous program driven by RAS activation.

We performed cell-line derived xenografts (CLDX) in NOD/SCID gamma-null (NSG) mice using TF-1-derived NF1-1, NF1-2 and CTR cell lines. Xenografted mice experienced AML-related symptoms within a median time of 28 days, 43 days and 76 days for NF1-1, NF1-2 and CTR groups, respectively (p<0.001 for comparison between NF1-depleted and control cells, data not shown). Leukemic cells mostly propagated into the bone marrow (data not shown), and also had a mild bloodstream diffusion (data not shown). Moreover, staining of bone marrow sections showed an increased ERK phosphorylation in mice transplanted with NF1 knockdown cells, in agreement with our in vitro observations (data not shown). Together these results suggest that NF1 genetic disruption and NRAS^{G12D} expression represent robust models for RAS/MAPK activation in AML.

Activity of MEK Inhibitors on RAS Pathway-Mutated AML.

We used the 592 compounds target selective inhibitor library (Selleck chemicals) mostly comprising kinase inhibitors to screen modified TF-1 cells at a 1004 concentration for each molecule. Strikingly, NF1 knockdown cells (NF1-1 and NF1-2 cell lines, cultured without GM-CSF) were more sensitive to MEK inhibitors compared to control cells (cultured with GM-CSF), while these cells were equally sensitive to p38 inhibitors (data not shown). While not included in the target inhibitor library, we further used the MEK inhibitor trametinib, currently developed in multiple clinical applications in oncology including in AML (13,45).

Dose-range experiments using three fold-dilutions of trametinib were carried out in RAS pathway activated TF-1, Ba/F3 and UT-7 cells, unambiguously showing that RAS activation correlated to a marked enhancement of trametinib cytotoxicity compared to isogenic control cells (data not shown). In TF-1 cells, trametinib markedly inhibited ERK phosphorylation since 0.5 h incubation, without affecting RAS activation (data not shown). Moreover, trametinib-induced cytotoxicity was associated with apoptosis induction, as shown by PARP and caspase-3 cleavage, and increased flow cytometry annexin V staining in NF1 knockdown TF-1 cells (data not shown). In another CLDX assay using a NF1-depleted TF-1 cell line, we observed that trametinib, given daily by oral gavage starting day 8 after transplant significantly prolonged mice survival (data not shown). From mice sacrificed 18 days after trametinib or vehicle onset, we showed that trametinib readily reached its target in vivo, as ERK phosphorylation was inhibited in bone marrow leukemic cells (data not shown). Together these data suggested that RAS activation induced an oncogenic addiction state, unmasking an exquisite sensitivity to the MEK inhibitor trametinib.

We performed colony-forming unit-leukemia (CFU-L) assays in 39 primary samples of AML patients from our cohort, incubated with vehicle or 50 nM trametinib, which significantly reduced the absolute number of CFU-L (p=0.0021, data not shown). Focusing on RAS pathway mutated samples, we observed that NRAS^{G12} and NRAS^{Q61R} mutated samples had a dramatic reduction of CFU-L formation in the presence of trametinib compared to other mutations (mostly KRAS and PTPN11 mutations) (data not shown). However, no difference in the formation of CFU-L was observed between NRAS^{G12} and NRAS^{Q61R} samples and those without RAS pathway mutation (data not shown). From the BEAT AML database (Tyner Nature 2018), we extracted cases matching with our patient's cohort by applying the same filters (data not shown). In contrast to the results of our CFU-L assays, we observed that RAS pathway mutated samples had a greater sensitivity to the MEK inhibitors trametinib and selumetinib compared to other samples in short-term liquid culture experiments, without significant difference between NRAS^{G12}/NRAS^{Q61R} and other RAS pathway mutated samples (data not shown). Our results suggested heterogeneous sensitivity to trametinib in CFU-L assays, with NRAS^{G12}/NRAS^{Q61R} mutated cases eliciting the best cytotoxic responses across RAS pathway mutated AML.

In fact, we treated an 84 years old woman for the transformation of a chronic myelomonocytic leukemia (CMML) to a NRAS^{G12A}-mutated AML (data not shown). At the AML stage, she first received 2000 mg daily hydroxycarbamide, which was switched for 2 mg/d trametinib after three weeks due to limited efficacy and hematological toxicity. During ten days of trametinib therapy, her white blood cell count (WBC) and monocyte count were at their lowest values. After trametinib discontinuation due to neurological side effects, WBC and monocyte count markedly increased. A second course of trametinib again dramatically reduced leukocytosis, before the definitive discontinuation of this molecule due to neurological toxicity (data not shown). Our patient unfortunately died from disease progression few days after therapeutic interruption. Interestingly, we observed a complete inhibition of ERK phosphorylation (data not shown), as well as a marked reduction of CFU-L formation (data not shown) with the leukemic cells from our patient incubated ex vivo with trametinib.

Collectively, these results suggested that RAS activated AML models were sensitive to the MEK inhibitor trametinib in vitro and in vivo, inhibiting ERK phosphorylation and promoting apoptosis. However, while of potential clinical interest, the activity of single agent trametinib appeared heterogeneous against AML patient samples.

Identification of Pyrvinium Pamoate as Potential New Agent in RAS Pathway Mutated AML.

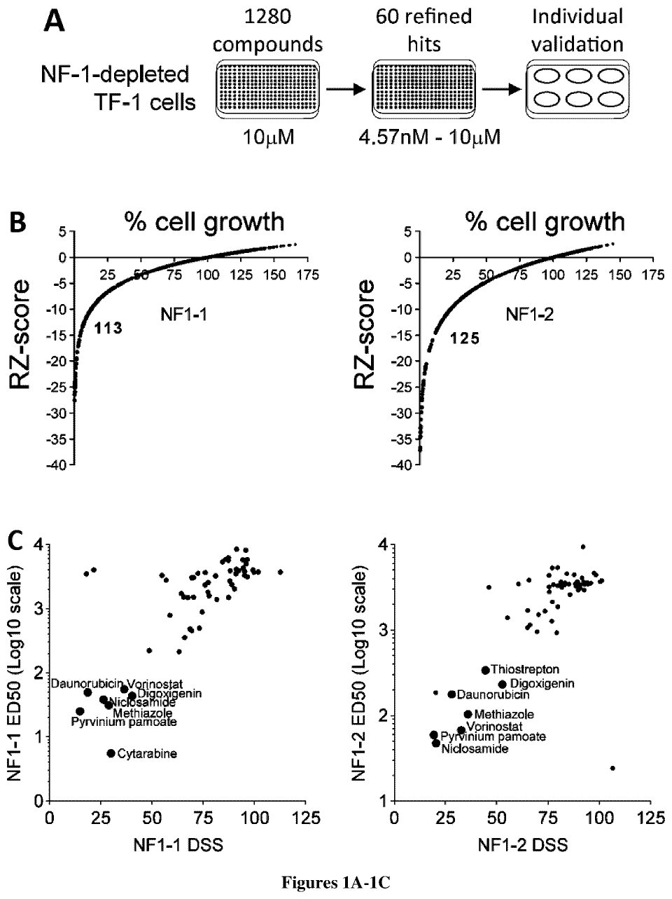

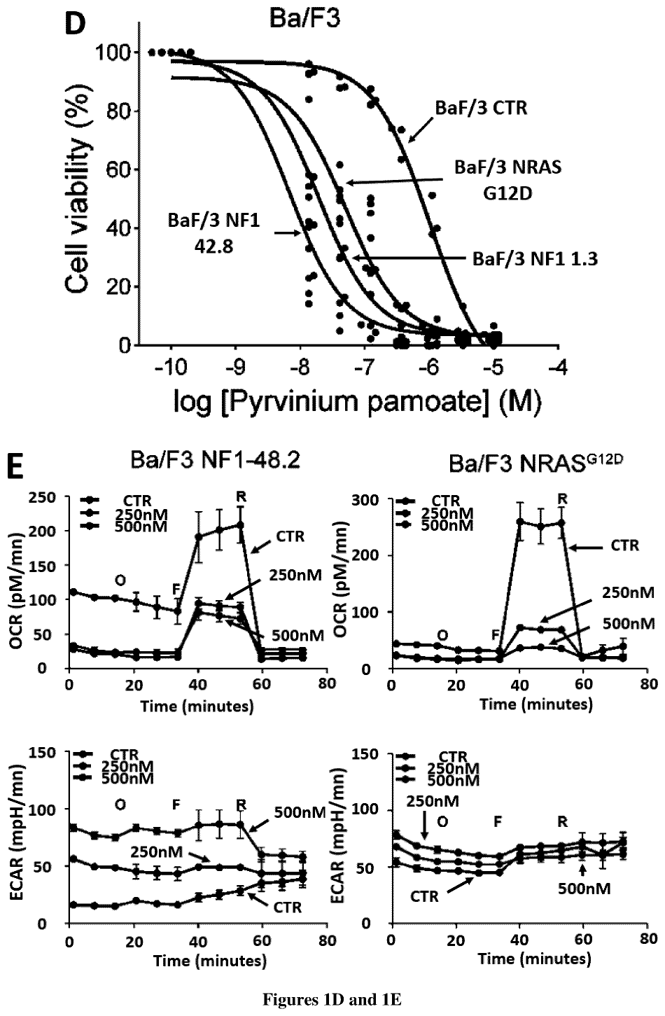

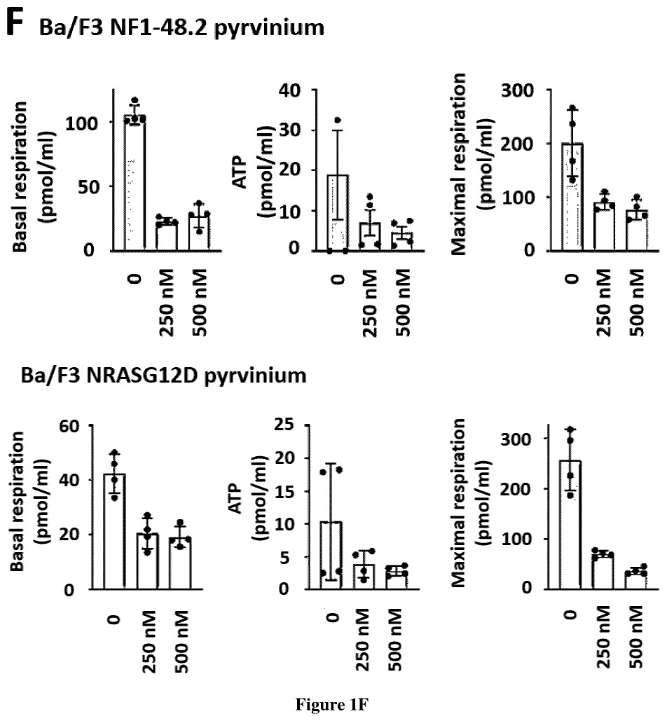

Building on our validated models of RAS activated AML, we performed a second pharmacological screen in a large library of 1280 FDA-approved molecules in a repurpose perspective (FIG. 1A). A first screen was performed at 10 μM for each compound in NF1 depleted TF-1 cell lines (NF1-1 and NF1-2). We selected 113 and 125 compounds having a RZ-score for cell growth inhibition ≤5 for NF1-1 and NF1-2 cell lines, respectively (FIG. 1B). We further refined these hits by filtrating redundant compounds (in terms of chemical family and/or pharmacodynamics), and we performed dose-range experiments (10⁻⁶ to 4.57×10⁻⁹ M) on the same cell lines with the top-60 compounds. Based on median dose-effect (effective dose 50, ED50) and drug sensitivity score (DSS), we selected six compounds as having the strongest cytotoxic activity on NF1-depleted cells, within the same range than the two key AML chemotherapies cytarabine and daunorubicin (FIG. 1C and data not shown). We assessed the activity of these six compounds individually in RAS-activated TF-1 and Ba/F3 cells to focus on a quinolone-derived molecule, pyrvinium pamoate (data not shown).

In activated RAS-dependent Ba/F3 cells, a minimal model of oncogene dependency widely employed in drug screening (38,46), pyrvinium pamoate dramatically decreased viability in NF1-depleted and NRAS^{G12D} mutated cells, compared to control cells (FIG. 1D). This RAS-dependent cytotoxicity was due to apoptosis induction, as shown in annexin V binding assays (data not shown). In NF1 knockdown AML cell lines, pyrvinium pamoate demonstrated a strong cytotoxic activity, but without sharp differences compared to control cell lines, possibly due to a significant RAS activation in cytokine-supplemented control cells (data not shown). In a panel of AML cell lines, pyrvinium pamoate generally demonstrated a greater cytotoxic potential in the presence of RAS pathway mutations (data not shown). Together these results suggested that pyrvinium pamoate preferentially targeted RAS mutated cells.

We aimed at understanding the molecular targets of pyrvinium pamoate in AML. First, we observed that pyrvinium pamoate inhibited ERK phosphorylation in NF1-depleted Ba/F3 cells, while this effect was moderate in TF-1 cells (data not shown). This discrepancy suggested that pyrvinium-induced cytotoxicity might not be a direct consequence of ERK/MAPK pathway inhibition. Pyrvinium pamoate may inhibit Wnt/β-catenin signaling in some models (47,48). We performed TOP/FOP Wnt signaling reporter assays and observed a near absence of Wnt activity in CTR, NF1-1, NF1-2 and NRAS^{G12D} TF-1 cells compared to the HUH-6 hepatoblastoma cell line (data not shown). Moreover, pyrvinium pamoate did not significantly inhibited Wnt activity in HUH-6 cells, although a trend to dose-dependent inhibition was observed (data not shown). We further assessed the potential impact of pyrvinium pamoate on mitochondrial respiration. We performed mitostress assays on NF1-depleted and NRAS^{G12D} Ba/F3 cells, incubated without or with pyrvinium pamaote and observed a decrease in basal and maximal respiration in pyrvinium-treated cells (FIGS. 1E-1F).

Altogether, agnostic screens identified pyrvinium pamoate as a preferentially cytotoxic drug in RAS-activated cells, potentially acting through mitochondrial respiration disruption.

Synergy Between the MEK Inhibitor Trametinib and Pyrvinium Pamoate in RAS Activated Cells.

While of potential therapeutic value, monotherapy by MEK inhibitor appeared to have a heterogeneous activity across RAS pathway mutated AML. Moreover, we learned from other models—particularly melanoma—that resistance mechanism acquisition to MEK inhibitors are common (14). Implementation of synergistic combinations with MEK inhibitors may thus represent an attractive therapeutic opportunity in RAS pathway mutated AML.

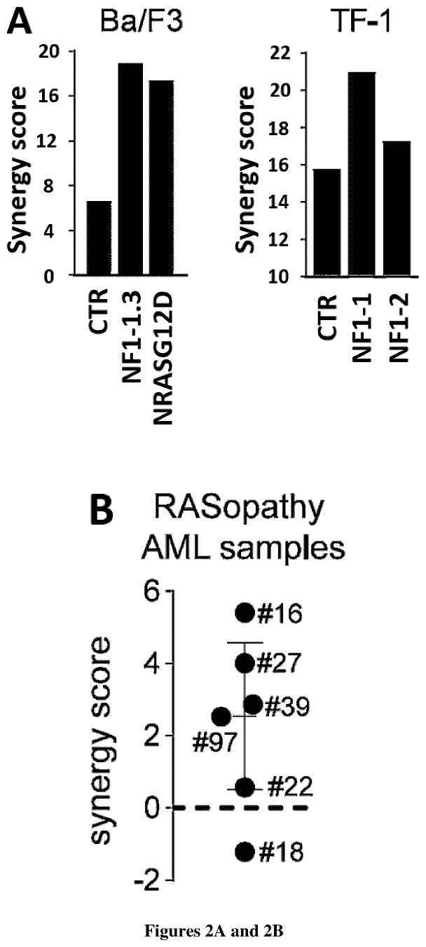

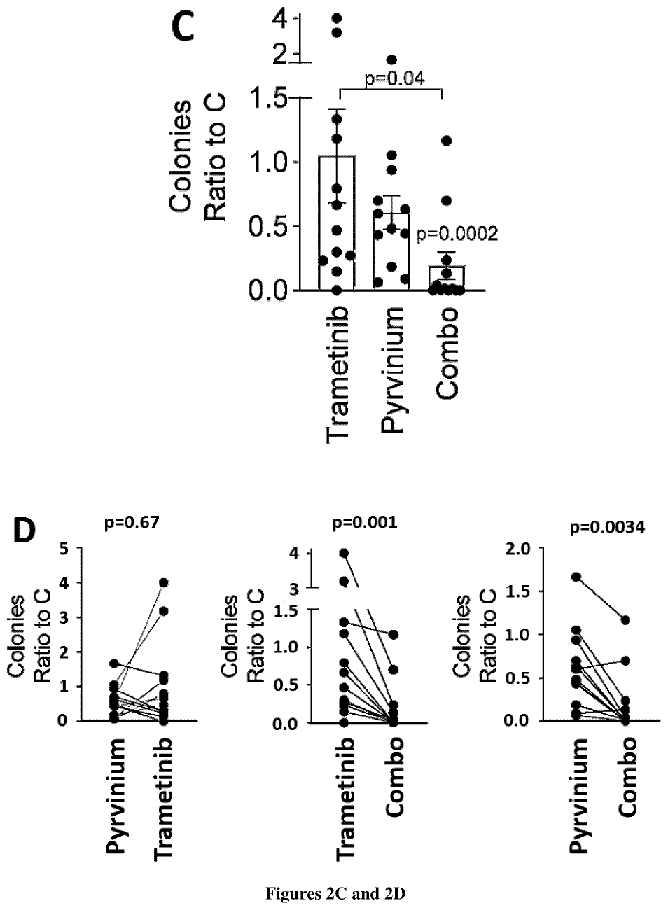

We thus combined trametinib and pyrvinium in dose-range cell viability assays in RAS-activated TF-1 and Ba/F3 cells. From cell viability crude data, we observed that trametinib and pyrvinium combination (combo) conditions had a markedly lower viability in RAS activated compared to control cells (Data not shown). Moreover, we calculated that RAS-activated cells had higher synergy scores compared to control cells, although this was less pronounced in TF-1 than in Ba/F3 cells (FIG. 2A). We performed similar short-term experiments in six RAS pathway mutated primary AML samples, and observed a synergy in four (FIG. 2B).

Compared to experiments performed in short-term liquid culture conditions, CFU-L assays allow the assessment of compound activity during longer periods (7 to 10 days), and on less mature AML progenitor cell populations (49). We performed CFU-L assays in 12 primary AML samples harboring RAS pathway mutations, incubated with vehicle, 50 nM trametinib, 250 nM pyrvinium or trametinib and pyrvinium combination (combo). While trametinib had no overall influence on colonies formation in these selected samples—and even increased colonies number in four cases—a trend to reduced CFU-L was observed with pyrvinium (FIG. 2C). Strikingly, a highly significant colonies formation inhibition was achieved by the combo compared to vehicle- or trametinib-treated cells (FIG. 2C). Interestingly, most samples resistant to single agents were sensitive to the other agent and had a dramatic CFU-L formation inhibition with trametinib and pyrvinium combo (FIG. 2D).

Experiments done in RAS-activated cell lines and primary AML samples thus suggested a robust synergistic activity of trametinib and pyrvinium with potential therapeutic applications.

### DISCUSSION

RAS was the first oncogene identified in human cancers, and its implication in oncogenesis has been widely studied since (8). While the genetic landscape of AML was solved these last few years, allowing the identification of molecular subgroups of patients with prognostic and/or therapeutic significance (7), RAS pathway mutations were barely considered as a particular entity. Recent studies unraveled frequent NRAS and KRAS mutations in core binding factors AML (CBFs, encompassing t(8;21) and inv(16) AML), and showed that the presence of RAS genes clonal interference discriminated between these seemingly good prognostic patients those having a reduced survival probability (50). Molecular mechanisms regulating the balance between activated RAS-GTP and inactive RAS-GDP are complex, involving multiple effectors such as protein kinases, scaffolding proteins, phosphatases, GAPs and GEFs (9). Mutations in genes encoding actors of this complex network are found in inherited genetic syndromes referred to as RASopathies genes (34). As somatic mutations of the same genes are reported in cancers, at a high frequency in the rare juvenile myelomonocytic leukemia (JMML), but also in as much as 25% of AML cases based on TCGA database (data not shown), we aimed at specifically considering RAS pathway altered AML from a descriptive, prognostic, and preclinical modeling and therapeutic perspective.

To focus on AML with unmet therapeutic needs, we excluded from our cohort AML cases of the favorable ELN prognostic group (t(15;17), t(8;21), inv(16), bi-allelic CEBPA, and NPM1 without FLT3-ITD mutations) (51). We also excluded FLT3-ITD cases due to mutual exclusion with RAS pathway mutations (data not shown). From a large AML cohort, we identified at least one RAS pathway alteration in 40% of our cases, which was higher than expected based on TCGA and BEAT AML cohorts, possibly due to the greater depth of our targeted NGS panel (average depth of 875 reads per base) (10,52). The most prevalent alterations concerned NF1, NRAS, KRAS and PTPN11, but we also observed variants affecting CBL, BRAF, RASA1, SOS1, MAP2K2 and RAF1 genes. Consistent with previous studies, we did not detect mutation within RAS pathways negative regulators including DUSP6, SPRED and SPRY family members (10,52). We observed 5.5% NF1 mutations, similar to previous studies (53-55), without cases harboring mutations at the potential Thr 676 hotspot reported by Eisfeld and colleagues (55). NF1 deletions, mostly found in case of complex chromosomal abnormalities, but also as cryptic FISH- and/or NGS-detected deletions were also frequent (9.5%). Finally, we observed concurrent NF1 and PRC2 members EZH2, EED and SUZ12 alterations, as reported in JMML (36,56). Interestingly, PRC2 disruption was reported to promote RAS-regulated genes transcription, cooperating with NF1 mutations in plexiform neurofibromas, glioma and melanoma (18). Moreover, Zhao and colleagues showed that concurrent Spry4 (a negative regulator of RAS), Nf1 and Tp53 deletions—as found in human AML with complex karyotype—represent leukemia initiating events in mouse models due to the additional loss of negative feedback on RAS (57). We may thus hypothesize that PRC2 alterations may amplify RAS oncogenic signal in NF1-altered AML.

While NRAS, KRAS, NF1 and PTPN11 mutations are generally reported as secondary driver events in AML, we observed different scenario based on VAFs analysis in some of our cases (2,11,58). Indeed, these mutations may be present in the main clone, suggesting an implication in early phases of disease onset, or in subclones. Moreover, 25% of RAS pathway mutated samples harbored two or more alterations of RAS genes. These alterations may be part of a single clone, supporting a dose-dependent effect of oncogenic RAS mutations as described in JMML (36), or may represent different populations with inter-clonal interference (11,50). Single-cell analysis of informative cases would be of major interest to better characterize the implication of RAS pathway mutations in leukemogenesis.

The clinical implications of RAS pathway alterations has not been considered globally so far. Several groups reported that NRAS and/or KRAS mutations lack prognostic significance in AML (35,59-62), with the possible exception of NRAS, DNMT3A and NPM1 co-mutated patients who may have favorable survival probabilities (7). Moreover, NF1 mutations were associated with reduced survival probability among the adverse cytogenetic subgroup of AML (55,63). In agreement with these data, NRAS or KRAS mutations had no impact on survival in our cohort. However, when considering RAS pathway alterations as a whole, this subgroup had a significantly reduced survival probability, particularly within the ELN intermediate prognostic group, in intensively treated AML patients. Interestingly, these mutations were significantly associated to increased leukemic cell proliferative markers including elevated white blood cell count, blast cell percentage and LDH levels. We also analyzed NF1 gene expression in a subset of our cases, and showed that low NF1 expression may correlate to a reduced survival probability among the RAS pathway alterations group, in agreement with the oncogenic cooperation observed between NF1 alterations and RAS mutations in mice models and human diseases (36,64). For these patients harboring wildtype NF1 alleles, other mechanisms may have accounted for NF1 downregulation such as promoter methylation (65) or NF1-targeting microRNA overexpression (66). Our data thus suggested considering RAS pathway altered AML cases as a provisional entity for prognostic and therapeutic research.

In a perspective of preclinical therapeutic development in RAS pathway altered AML, we implemented robust cell lines models of RAS activation. We took advantage of growth-factor dependent cell lines (TF-1, Ba/F3 and UT-7) to assess the oncogenic potential of RAS-activating genetic alterations. Using CRISPR/Cas9, we depleted NF1 from these cell lines, which demonstrated cytokine-independency, in agreement with the observations made in an Nf1 knockout murine fetal liver cells model (67), and RAS pathway oncogenic addiction demonstrated by exquisite sensitivity to MEK inhibitors in vitro, and in vivo in mice CLDX experiments. Similar results were achieved using NRAS^{G12D} expression in these cells, as reported (68). However, even if the clinically-used MEK inhibitor trametinib exhibited anti-leukemic activity against primary AML samples ex vivo and demonstrated clinical-grade activity in a NRAS^{G12D}-mutated AML patient lacking other therapeutic perspective, its activity was heterogeneous and mostly seen in NRAS-mutated samples. In other RAS pathway cases, particularly those harboring PTPN11 mutations, we observed a low efficiency and even an increased proliferation and survival induced by trametinib, suggesting the development of bypass mechanisms to single-agent MEK inhibition in these cases, as reported (14). From the BEAT AML database (52), we found in contrast that MEK inhibitor had a significantly higher cytotoxic activity in RAS altered samples—and not electively in NRAS-mutated cases—in short-term assays, which suggested that resistance mechanisms to MEK inhibition might require longer periods to occur in vitro.

The heterogeneity of RAS pathway mutations and the complexity of their biological consequences in cancer cells (9,69) suggest the development of combinatorial therapies to overcome preexisting or acquired resistance mechanisms to RAS-dependent pathways inhibition (70). Following a repurposing strategy to find new AML drugs with potential activity in RAS pathway altered cases, we screened NF1 knockdown AML cells from the TF-1 cell line using a large FDA-approved molecules library (71). These experiments led to the identification of pyrvinium pamoate, an oral anthelminthic drug employed in pinworm infection (72). This compound exerted a strong cytotoxic activity against different RAS-mutated models, and in primary AML samples ex vivo. Interestingly, pyrvinium had a preferential cytotoxicity against RAS-activated Ba/F3 cells and appeared slightly more active against RAS-mutated AML cell lines. Several mechanisms of action of pyrvinium were described, including the inhibition of Wnt/β-catenin pathway in different cancer types (47,48,73). While we ruled out Wnt inhibition by pyrvinium pamoate in our models in vitro, we focused on a potential metabolic activity of pyrvinium pamoate. Indeed, we found that this molecule dose-dependently inhibited mitochondrial respiration, in agreement with observations made in other cancers and in FLT3-mutated AML (74-77). We further observed a strong synergy between trametinib and pyrvinium in RAS-activated cell lines models, but also in primary samples from AML patients, in short-term liquid culture experiments and in long-term CFU-L assays. Interestingly, RAS activation may orchestrate cancer cells energetic metabolic reprogramming such as a shift toward glycolysis (78-80) or diversion of glycolysis intermediates into anabolic pathways (78). In fact, MEK inhibitors may reverse a RAS-driven glycolysis phenotype toward increased mitochondrial respiration (80,81). We could hypothesize that in RAS activated AML, trametinib may inhibits RAS-induced glycolysis shift, inducing a dependency to mitochondrial respiration thereby opening a therapeutic window for respiratory chain-targeting compounds.

Direct pharmacological targeting of activated RAS remains one of the most challenging problem of cancer drug discovery, although recent advances appeared promising for subsets of patients including those harboring KRAS^{G12c} (82) and semi-autonomous RAS pathway activation or PTPN11 mutations (83-85). As we showed that RAS pathway mutated AML patients, currently lacking personalized therapies in contrast to other AML subtypes had an adverse outcome upon conventional AML therapies, we suggest that the clinical development of pyrvinium pamoate may represent a meaningful opportunity in RAS pathway mutated AML.

TABLE 1
Clinical characteristics of RAS pathway mutated patients compared to other patients

| | RAS pathway | | No RAS pathway | | |
|---|---|---|---|---|---|
| | alterations | | alterations | | P value |
| Number of Patients | 34 | | 45 | | |
| Gender (Male) n (%) | 22 | (65%) | 29 | (64%) | p > 0.999 |
| Age (years) | 61 | (23-78) | 31 | (26-82) | p = 0.9431 |
| White Blood Count (G/L) | 19.5 | (1.1-287) | 2.3 | (0.8-170) | p < 0.0002 |
| Missing data | 1 | | 1 | | |
| Bone marrow blast percentage (%) | 62 | (22-94) | 44 | (7-98) | p = 0.0097 |
| Missing data | 1 | | 3 | | |
| Peripheral blast percentage (%) | 50 | (0-98) | 4.5 | (0-97) | p < 0.0001 |
| Missing data | 1 | | 3 | | |
| LDH (IU/L) | 501 | (150-3516) | 347 | (56-18000) | p = 0.0236 |
| Missing data | 0 | | 3 | | |
| Allogeneic Stem Cell | 8 | (24%) | 13 | (32%) | p = 0.4536 |
| Transplantation, n (%) | | | | | |
| Missing data | 0 | | 4 | | |
| Secondary Disease, n (%) | 6 | (17.6%) | 6 | (13%) | p = 0.7534 |
| Refractory Disease, n (%) | 13 | (43%) | 17 | (39%) | p = 1 |

| | | |
|---|---|---|
| Not evaluable | 4 | 2 |

### REFERENCES

1. Bullinger L, Döhner K, Döhner H. Genomics of Acute Myeloid Leukemia Diagnosis and Pathways. J Clin Oncol Off J Am Soc Clin Oncol. 20 mars 2017; 35(9):934-46.
2. Tuval A, Shlush L I. Evolutionary trajectory of leukemic clones and its clinical implications. Haematologica. mai 2019; 104(5):872-80.
3. Heuser M, Mina A, Stein E M, Altman J K. How Precision Medicine Is Changing Acute Myeloid Leukemia Therapy. Am Soc Clin Oncol Educ Book Am Soc Clin Oncol Annu Meet. janv 2019; 39:411-20.
4. Stone R M, Mandrekar S J, Sanford B L, Laumann K, Geyer S, Bloomfield C D, et al. Midostaurin plus Chemotherapy for Acute Myeloid Leukemia with a FLT3 Mutation. N Engl J Med. March 2017; 377(5):454-64.
5. Stein E M, DiNardo C D, Fathi A T, Pollyea D A, Stone R M, Altman J K, et al. Molecular remission and response patterns in patients with mutant-IDH2 acute myeloid leukemia treated with enasidenib. Blood. 14 févr 2019; 133(7):676-87.
6. Pollyea D A, Tallman M S, de Botton S, Kantarjian H M, Collins R, Stein A S, et al. Enasidenib, an inhibitor of mutant IDH2 proteins, induces durable remissions in older patients with newly diagnosed acute myeloid leukemia. Leukemia. 9 avr 2019;
7. Papaemmanuil E, Gerstung M, Bullinger L, Gaidzik V I, Paschka P, Roberts N D, et al. Genomic Classification and Prognosis in Acute Myeloid Leukemia. N Engl J Med. 9 juin 2016; 374(23):2209-21.
8. Pylayeva-Gupta Y, Grabocka E, Bar-Sagi D. RAS oncogenes: weaving a tumorigenic web. Nat Rev Cancer. 13 Oct. 2011; 11(11):761-74.
9. Simanshu D K, Nissley D V, McCormick F. RAS Proteins and Their Regulators in Human Disease. Cell. 29 juin 2017; 170(1):17-33.
10. Cancer Genome Atlas Research Network. Genomic and epigenomic landscapes of adult de novo acute myeloid leukemia. N Engl J Med. 30 mai 2013; 368(22):2059-74.
11. Hirsch P, Zhang Y, Tang R, Joulin V, Boutroux H, Pronier E, et al. Genetic hierarchy and temporal variegation in the clonal history of acute myeloid leukaemia. Nat Commun. 18 2016; 7:12475.
12. Burgess M R, Hwang E, Firestone A J, Huang T, Xu J, Zuber J, et al. Preclinical efficacy of MEK inhibition in Nras-mutant AML. Blood. 18 déc. 2014; 124(26):3947-55.
13. Borthakur G, Popplewell L, Boyiadzis M, Foran J, Platzbecker U, Vey N, et al. Activity of the oral mitogen-activated protein kinase kinase inhibitor trametinib in RAS-mutant relapsed or refractory myeloid malignancies. Cancer. 15 2016; 122(12):1871-9.
14. Caunt C J, Sale M J, Smith P D, Cook S J. MEK1 and MEK2 inhibitors and cancer therapy: the long and winding road. Nat Rev Cancer. 2015; 15(10):577-92.
15. Stephen A G, Esposito D, Bagni R K, McCormick F. Dragging ras back in the ring. Cancer Cell. 17 mars 2014; 25(3):272-81.
16. Ryan M B, Corcoran R B. Therapeutic strategies to target RAS-mutant cancers. Nat Rev Clin Oncol. 2018; 15(11):709-20.
17. Sujobert P, Poulain L, Paubelle E, Zylbersztejn F, Grenier A, Lambert M, et al. Co-activation of AMPK and mTORC1 Induces Cytotoxicity in Acute Myeloid Leukemia. Cell Rep. 9 juin 2015; 11(9):1446-57.
18. De Raedt T, Beert E, Pasmant E, Luscan A, Brems H, Ortonne N, et al. PRC2 loss amplifies Ras-driven transcription and confers sensitivity to BRD4-based therapies. Nature. 9 Oct. 2014; 514(7521):247-51.
19. Pasmant E, Parfait B, Luscan A, Goussard P, Briand-Suleau A, Laurendeau I, et al. Neurofibromatosis type 1 molecular diagnosis: what can NGS do for you when you have a large gene with loss of function mutations? Eur J Hum Genet EJHG. mai 2015; 23(5):596-601.
20. Romana S P, Radford-Weiss I, Ben Abdelali R, Schluth C, Petit A, Dastugue N, et al. NUP98 rearrangements in hematopoietic malignancies: a study of the Groupe Francophone de Cytogénétique Hématologique. Leukemia. avr 2006; 20(4):696-706.
21. Hospital M-A, Jacquel A, Mazed F, Saland E, Larrue C, Mondesir J, et al. RSK2 is a new Pim2 target with pro-survival functions in FLT3-ITD-positive acute myeloid leukemia. Leukemia. 2018; 32(3):597-605.
22. Shalem O, Sanjana N E, Hartenian E, Shi X, Scott D A, Mikkelson T, et al. Genome-scale CRISPR-Cas9 knockout screening in human cells. Science. 3 janv 2014; 343(6166):84-7.
23. Campeau E, Ruhl V E, Rodier F, Smith C L, Rahmberg B L, Fuss J O, et al. A versatile viral system for expression and depletion of proteins in mammalian cells. PloS One. 6 août 2009; 4(8):e6529.
24. Grenier A, Sujobert P, Olivier S, Guermouche H, Mondésir J, Kosmider O, et al. Knockdown of Human AMPK Using the CRISPR/Cas9 Genome-Editing System. Methods Mol Biol Clifton N.J. 2018; 1732:171-94.
25. Saland E, Boutzen H, Castellano R, Pouyet L, Griessinger E, Larrue C, et al. A robust and rapid xenograft model to assess efficacy of chemotherapeutic agents for human acute myeloid leukemia. Blood Cancer J. 20 mars 2015; 5:e297.
26. Malo N, Hanley J A, Cerquozzi S, Pelletier J, Nadon R. Statistical practice in high-throughput screening data analysis. Nat Biotechnol. févr 2006; 24(2):167-75.
27. Birmingham A, Selfors L M, Forster T, Wrobel D, Kennedy C J, Shanks E, et al. Statistical methods for analysis of high-throughput RNA interference screens. Nat Methods. août 2009; 6(8):569-75.
28. Ritz C, Baty F, Streibig J C, Gerhard D. Dose-Response Analysis Using R. PloS One. 2015; 10(12):e0146021.
29. Yadav B, Pemovska T, Szwajda A, Kulesskiy E, Kontro M, Karjalainen R, et al. Quantitative scoring of differential drug sensitivity for individually optimized anticancer therapies. Sci Rep. 5 juin 2014; 4:5193.
30. Poulain L, Sujobert P, Zylbersztejn F, Barreau S, Stuani L, Lambert M, et al. High mTORC1 activity drives glycolysis addiction and sensitivity to G6PD inhibition in acute myeloid leukemia cells. Leukemia. 2017; 31(11):2326-35.
31. Korinek V, Barker N, Morin P J, van Wichen D, de Weger R, Kinzler K W, et al. Constitutive transcriptional activation by a beta-catenin-Tcf complex in APC−/− colon carcinoma. Science. 21 mars 1997; 275(5307):1784-7.
32. Tamburini J, Green A S, Bardet V, Chapuis N, Park S, Willems L, et al. Protein synthesis is resistant to rapamycin and constitutes a promising therapeutic target in acute myeloid leukemia. Blood. 20 août 2009; 114(8):1618-27.
33. Ianevski A, He L, Aittokallio T, Tang J. SynergyFinder: a web application for analyzing drug combination dose-response matrix data. Bioinformatics. 1 août 2017; 33(15):2413-5.
34. Tajan M, Paccoud R, Branka S, Edouard T, Yart A. The RASopathy Family: Consequences of Germline Activation of the RAS/MAPK Pathway. Endocr Rev. 1 Oct. 2018; 39(5):676-700.
35. Bacher U, Haferlach T, Schoch C, Kern W, Schnittger S. Implications of NRAS mutations in AML: a study of 2502 patients. Blood. 15 mai 2006; 107(10):3847-53.
36. Caye A, Strullu M, Guidez F, Cassinat B, Gazal S, Fenneteau O, et al. Juvenile myelomonocytic leukemia displays mutations in components of the RAS pathway and the PRC2 network. Nat Genet. nov 2015; 47(11):1334-40.
37. Büchner T, Schlenk R F, Schaich M, Döhner K, Krahl R, Krauter J, et al. Acute Myeloid Leukemia (AML): different treatment strategies versus a common standard arm-combined prospective analysis by the German A M L Intergroup. J Clin Oncol Off J Am Soc Clin Oncol. 10 Oct. 2012; 30(29):3604-10.
38. Warmuth M, Kim S, Gu X, Xia G, Adrian F. Ba/F3 cells and their use in kinase drug discovery. Curr Opin Oncol. janv 2007; 19(1):55-60.
39. Wang F, Travins J, DeLaBarre B, Penard-Lacronique V, Schalm S, Hansen E, et al. Targeted inhibition of mutant IDH2 in leukemia cells induces cellular differentiation. Science. 3 mai 2013; 340(6132):622-6.
40. Palacios R, Henson G, Steinmetz M, McKearn J P. Interleukin-3 supports growth of mouse pre-B-cell clones in vitro. Nature. 10 mai 1984; 309(5964):126-31.
41. Palacios R, Steinmetz M. Il-3-dependent mouse clones that express B-220 surface antigen, contain Ig genes in germ-line configuration, and generate B lymphocytes in vivo. Cell. juill 1985; 41(3):727-34.
42. Mootha V K, Lindgren C M, Eriksson K-F, Subramanian A, Sihag S, Lehar J, et al. PGC-1 alpha-responsive genes involved in oxidative phosphorylation are coordinately downregulated in human diabetes. Nat Genet. juill 2003; 34(3):267-73.
43. Subramanian A, Tamayo P, Mootha V K, Mukherjee S, Ebert B L, Gillette M A, et al. Gene set enrichment analysis: a knowledge-based approach for interpreting genome-wide expression profiles. Proc Natl Acad Sci USA. 25 Oct. 2005; 102(43):15545-50.
44. Chrétien S, Varlet P, Verdier F, Gobert S, Cartron J P, Gisselbrecht S, et al. Erythropoietin-induced erythroid differentiation of the human erythroleukemia cell line TF-1 correlates with impaired STATS activation. EMBO J. 15 août 1996; 15(16):4174-81.
45. Long G V, Hauschild A, Santinami M, Atkinson V, MandalàM, Chiarion-Sileni V, et al. Adjuvant Dabrafenib plus Trametinib in Stage III BRAF-Mutated Melanoma. N Engl J Med. September 2017; 377(19):1813-23.
46. Oetzel C, Jonuleit T, Götz A, van der Kuip H, Michels H, Duyster J, et al. The tyrosine kinase inhibitor CGP 57148 (ST1 571) induces apoptosis in BCR-ABL-positive cells by down-regulating BCL-X. Clin Cancer Res Off J Am Assoc Cancer Res. mai 2000; 6(5):1958-68.
47. Xu W, Lacerda L, Debeb B G, Atkinson R L, Solley T N, Li L, et al. The antihelmintic drug pyrvinium pamoate targets aggressive breast cancer. PloS One. 2013; 8(8):e71508.
48. Thorne C A, Hanson A J, Schneider J, Tahinci E, Orton D, Cselenyi C S, et al. Small-molecule inhibition of Wnt signaling through activation of casein kinase 1α. Nat Chem Biol. nov 2010; 6(11):829-36.
49. Récher C, Beyne-Rauzy O, Demur C, Chicanne G, Dos Santos C, Mas VM-D, et al. Antileukemic activity of rapamycin in acute myeloid leukemia. Blood. 15 mars 2005; 105(6):2527-34.
50. Itzykson R, Duployez N, Fasan A, Decool G, Marceau-Renaut A, Meggendorfer M, et al. Clonal interference of signaling mutations worsens prognosis in core-binding factor acute myeloid leukemia. Blood. 12 juill 2018; 132(2):187-96.
51. Döhner H, Estey E, Grimwade D, Amadori S, Appelbaum F R, Büchner T, et al. Diagnosis and management of AML in adults: 2017 ELN recommendations from an international expert panel. Blood. 26 2017; 129(4):424-47.
52. Tyner J W, Tognon C E, Bottomly D, Wilmot B, Kurtz S E, Savage S L, et al. Functional genomic landscape of acute myeloid leukaemia. Nature. 2018; 562(7728):526-31.
53. Haferlach C, Grossmann V, Kohlmann A, Schindela S, Kern W, Schnittger S, et al. Deletion of the tumor-suppressor gene NF1 occurs in 5% of myeloid malignancies and is accompanied by a mutation in the remaining allele in half of the cases. Leukemia. avr 2012; 26(4):834-9.
54. Boudry-Labis E, Roche-Lestienne C, Nibourel O, Boissel N, Terre C, Perot C, et al. Neurofibromatosis-1 gene deletions and mutations in de novo adult acute myeloid leukemia. Am J Hematol. avr 2013; 88(4):306-11.
55. Eisfeld A-K, Kohlschmidt J, Mrózek K, Mims A, Walker C J, Blachly J S, et al. NF1 mutations are recurrent in adult acute myeloid leukemia and confer poor outcome. Leukemia. déc 2018; 32(12):2536-45.
56. Stieglitz E, Taylor-Weiner A N, Chang T Y, Gelston L C, Wang Y-D, Mazor T, et al. The genomic landscape of juvenile myelomonocytic leukemia. Nat Genet. nov 2015; 47(11):1326-33.
57. Zhao Z, Chen C-C, Rillahan C D, Shen R, Kitzing T, McNerney M E, et al. Cooperative loss of RAS feedback regulation drives myeloid leukemogenesis. Nat Genet. mai 2015; 47(5):539-43.
58. Corces-Zimmerman M R, Hong W-J, Weissman I L, Medeiros B C, Majeti R. Preleukemic mutations in human acute myeloid leukemia affect epigenetic regulators and persist in remission. Proc Natl Acad Sci USA. 18 févr 2014; 111(7):2548-53.
59. Bowen D T, Frew M E, Hills R, Gale R E, Wheatley K, Groves M J, et al. RAS mutation in acute myeloid leukemia is associated with distinct cytogenetic subgroups but does not influence outcome in patients younger than 60 years. Blood. 15 sept 2005; 106(6):2113-9.
60. Ritter M, Kim T D, Lisske P, Thiede C, Schaich M, Neubauer A. Prognostic significance of N-RAS and K-RAS mutations in 232 patients with acute myeloid leukemia. Haematologica. nov 2004; 89(11):1397-9.
61. Illmer T, Thiede C, Fredersdorf A, Stadler S, Neubauer A, Ehninger G, et al. Activation of the RAS pathway is predictive for a chemosensitive phenotype of acute myelogenous leukemia blasts. Clin Cancer Res Off J Am Assoc Cancer Res. 1 mai 2005; 11(9):3217-24.
62. Liu X, Ye Q, Zhao X-P, Zhang P-B, Li S, Li R-Q, et al. RAS mutations in acute myeloid leukaemia patients: A review and meta-analysis. Clin Chim Acta Int J Clin Chem. févr 2019; 489:254-60.
63. Moison C, Lavallée V-P, Thiollier C, Lehnertz B, Boivin I, Mayotte N, et al. Complex karyotype AML displays G2/M signature and hypersensitivity to PLK1 inhibition. Blood Adv. 26 févr 2019; 3(4):552-63.
64. Cutts B A, Sjogren A-KM, Andersson K M E, Wahlstrom A M, Karlsson C, Swolin B, et al. Nf1 deficiency cooperates with oncogenic K-RAS to induce acute myeloid leukemia in mice. Blood. 22 Oct. 2009; 114(17):3629-32.
65. Yang G, Khalaf W, van de Locht L, Jansen J H, Gao M, Thompson M A, et al. Transcriptional repression of the Neurofibromatosis-1 tumor suppressor by the t(8;21) fusion protein. Mol Cell Biol. juill 2005; 25(14):5869-79.
66. Lenarduzzi M, Hui A B Y, Alajez N M, Shi W, Williams J, Yue S, et al. MicroRNA-193b enhances tumor progression via down regulation of neurofibromin 1. PloS One. 2013; 8(1):e53765.
67. Donovan S, See W, Bonifas J, Stokoe D, Shannon K M. Hyperactivation of protein kinase B and ERK have discrete effects on survival, proliferation, and cytokine expression in Nf1-deficient myeloid cells. Cancer Cell. 1 déc. 2002; 2(6):507-14.
68. Nonami A, Sattler M, Weisberg E, Liu Q, Zhang J, Patricelli M P, et al. Identification of novel therapeutic targets in acute leukemias with NRAS mutations using a pharmacologic approach. Blood. 14 mai 2015; 125(20):3133-43.
69. Li S, Balmain A, Counter C M. A model for RAS mutation patterns in cancers: finding the sweet spot. Nat Rev Cancer. 2018; 18(12):767-77.
70. Al-Lazikani B, Banerji U, Workman P. Combinatorial drug therapy for cancer in the post-genomic era. Nat Biotechnol. 10 juill 2012; 30(7):679-92.
71. Bertolini F, Sukhatme V P, Bouche G. Drug repurposing in oncology-patient and health systems opportunities. Nat Rev Clin Oncol. déc 2015; 12(12):732-42.
72. Momtazi-Borojeni A A, Abdollahi E, Ghasemi F, Caraglia M, Sahebkar A. The novel role of pyrvinium in cancer therapy. J Cell Physiol. 2018; 233(4):2871-81.
73. Chen B, Ma L, Paik H, Sirota M, Wei W, Chua M-S, et al. Reversal of cancer gene expression correlates with drug efficacy and reveals therapeutic targets. Nat Commun. December 2017; 8:16022.
74. Xiang W, Cheong J K, Ang S H, Teo B, Xu P, Asari K, et al. Pyrvinium selectively targets blast phase-chronic myeloid leukemia through inhibition of mitochondrial respiration. Oncotarget. 20 Oct. 2015; 6(32):33769-80.
75. Harada Y, Ishii I, Hatake K, Kasahara T. Pyrvinium pamoate inhibits proliferation of myeloma/erythroleukemia cells by suppressing mitochondrial respiratory complex I and STAT3. Cancer Lett. 1 juin 2012; 319(1):83-8.
76. Xiao M, Zhang L, Zhou Y, Rajoria P, Wang C. Pyrvinium selectively induces apoptosis of lymphoma cells through impairing mitochondrial functions and JAK2/STATS. Biochem Biophys Res Commun. 15 janv 2016; 469(3):716-22.
77. Fu Y-H, Lu W-H, Lan P-Q, Hu C-Y, Chen C-Y, Ou D-L, et al. Pyrvinium Pamoate Overcomes Cabozantinib-Resistance of FLT3-ITD AML Cells through Modulating the Mitochondria Functions and Signaling Pathways. Blood. 21 Nov. 2018; 132 (Suppl 1):4683-4683.
78. Ying H, Kimmelman A C, Lyssiotis C A, Hua S, Chu G C, Fletcher-Sananikone E, et al. Oncogenic Kras maintains pancreatic tumors through regulation of anabolic glucose metabolism. Cell. 27 avr 2012; 149(3):656-70.
79. Yun J, Rago C, Cheong I, Pagliarini R, Angenendt P, Rajagopalan H, et al. Glucose deprivation contributes to the development of KRAS pathway mutations in tumor cells. Science. 18 Sep. 2009; 325(5947):1555-9.
80. Haq R, Shoag J, Andreu-Perez P, Yokoyama S, Edelman H, Rowe G C, et al. Oncogenic BRAF regulates oxidative metabolism via PGC1α and MITF. Cancer Cell. 18 mars 2013; 23(3):302-15.
81. Serasinghe M N, Gelles J D, Li K, Zhao L, Abbate F, Syku M, et al. Dual suppression of inner and outer mitochondrial membrane functions augments apoptotic responses to oncogenic MAPK inhibition. Cell Death Dis. 18 2018; 9(2):29.
82. Ostrem J M, Peters U, Sos M L, Wells J A, Shokat K M. K-Ras(G12C) inhibitors allosterically control GTP affinity and effector interactions. Nature. 28 Nov. 2013; 503(7477):548-51.
83. Mainardi S, Mulero-Sánchez A, Prahallad A, Germano G, Bosma A, Krimpenfort P, et al. SHP2 is required for growth of KRAS-mutant non-small-cell lung cancer in vivo. Nat Med. 2018; 24(7):961-7.
84. Wong G S, Zhou J, Liu J B, Wu Z, Xu X, Li T, et al. Targeting wild-type KRAS-amplified gastroesophageal cancer through combined MEK and SHP2 inhibition. Nat Med. 2018; 24(7):968-77.
85. Nichols R J, Haderk F, Stahlhut C, Schulze C J, Hemmati G, Wildes D, et al. RAS nucleotide cycling underlies the SHP2 phosphatase dependence of mutant BRAF-, NF1- and RAS-driven cancers. Nat Cell Biol. 2018; 20(9):1064-73.

Throughout this application, various references describe the state of the art to which this invention pertains. The disclosures of these references are hereby incorporated by reference into the present disclosure.

1. A method of treating a RAS pathway mutated acute myeloid leukemia and/or treating a RAS pathway mutated acute myeloid leukemia resistant to MEK inhibitors in a patient in need thereof comprising administering to the patient a therapeutically effective amount of pyrvinium.

2. A method of treating a RAS pathway mutated acute myeloid leukemia in a patient in need thereof and/or enhancing the potency of a MEK inhibitor administered to a subject suffering from a RAS pathway mutated acute myeloid leukemia as part of a treatment regimen, comprising administering to the subject a therapeutically effective combination comprising MEK inhibitor and pyrvinium.

3. (canceled)

4. (canceled)

5. A method of preventing resistance to an administered MEK inhibitor in a subject suffering from a RAS pathway mutated acute myeloid leukemia comprising administering to the subject a therapeutically effective amount of pyrvinium.

6. The method according to claim 1, wherein the patient harbors at least one mutation in at least one gene selected from the group consisting of RAS, NRAS, KRAS, NF1, PTPN11, BRAF, CBL, RASA1, RAF1, SOS1, and MAP2K2.

7. The method according to claim 2, wherein the MEK inhibitor is trametinib.

8. The method according to claim 2, wherein the patient harbors at least one mutation in at least one gene selected from the group consisting of RAS, NRAS, KRAS, NF1, PTPN11, BRAF, CBL, RASA1, RAF1, SOS1, and MAP2K2.
